SEQUENCE LISTING (1 sequence)

SEQ ID NO: 1 - protein, 502 aa, Artificial sequence
  Amino acid sequnce of Extracellular Domain of TLT2-Immunoglobulin Hinge-Fc region
MEPWPLTFLLLLLLLLWLQGCVSGHSNENLYRKVWRREGETLSVQCSYKNRRNLVEAKSWCKVKKKKCDHNFTRSWVRGPSYSLRDDAKVKVVRITMEALRVQDSGRYWCMRNTAGHFYPLVGFQLEVYPALTTERNVPHTHLTNTPMDGFVTTGQVHISDPHAPFTSDVTMFTSEVTMFTSGLLTLASGTTTPTPVTGYSFIDTSGTVTEPERNTESQPATLSPSNARSFSADPVTTSTMSRHQSSSLSTTGTCHPLTPNRSQETYSPGELKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2013/0216540 A1
(43) Pub. Date: Aug. 22, 2013

(54) MODULATION OF THE INNATE IMMUNE SYSTEM THROUGH THE TREM-LIKE TRANSCRIPT 2 PROTEIN

(71) Applicant: The UAB Research Foundation, (US)

(72) Inventors: Louis B. Justement, Birmingham, AL (US); Rodney G. King, Fultondale, AL (US)

(73) Assignee: THE UAB RESEARCH FOUNDATION, Birmingham, AL (US)

(21) Appl. No.: 13/773,039

(22) Filed: Feb. 21, 2013

Related U.S. Application Data
(60) Provisional application No. 61/601,639, filed on Feb. 22, 2012.

(51) Int. Cl. A61K 39/395; A61K 38/17

(52) U.S. Cl. CPC A61K 39/3955; A61K 38/1774
USPC 424/134.1; 424/173.1; 424/143.1; 435/375; 514/44 R

(57) ABSTRACT

TREM-like transcript 2 (TLT2), is expressed on neutrophils, macrophages, and B lymphocytes. Expression of TLT2 is up-regulated on neutrophils and macrophages in response to inflammatory stimuli in vivo and synergizes with agonists that bind to G-protein coupled receptors (GPCR) to potentiate the neutrophil antibacterial and chemotactic response. Administration of anti-TLT2 mAb enhances the acute inflammatory response in vivo that is associated with increased neutrophil recruitment to sites of inflammation. TLT2 ligation in vivo also potentiates chemokine and growth factor production indicating that TLT2 can exert both neutrophil intrinsic and extrinsic effects. The administration of anti-TLT2 mAb alone promotes neutrophil recruitment to the lung and peritoneum, as well as the rapid production of G-CSF, CXCL1 (KC) and CXCL2 (MIP-2). Additionally, the administration of an agent to the circulatory system of an animal can reduce the availability of a TLT2 endogenous ligand to reduce the extent of a neutrophil or macrophage-induced inflammatory response.

### CROSS-REFERENCE TO RELATED APPLICATIONS

This application claims priority to U.S. Provisional Patent Application Ser. No. 61/601,639 entitled “TREM-LIKE TRANSCRIPT 2 MONOCLONAL ANTIBODIES AND RECOMBINANT PROTEINS” filed Feb. 22, 2012, the entirety of which is hereby incorporated by reference.

### STATEMENT REGARDING FEDERALLY SPONSORED RESEARCH OR DEVELOPMENT

This invention was made with government support under Institutional Training in Immunology Grant No. NIH (T32 AI 007051-33) awarded by the National institutes of Health of the United States government. The government has certain rights in the invention.

### SEQUENCE LISTING

The present disclosure includes a sequence listing incorporated herein by reference in its entirety.

### TECHNICAL FIELD

The present disclosure is generally related to methods of modulating the activity of the innate immune system. The disclosure further relates to modulating the response of neutrophils and macrophages to such as an infection of an animal or human by ligation of a TREM-like transcript-2 cell protein. The disclosure relates still further to methods of modulating an inflammatory response in an animal or human by down-regulating the availability of TREM-like transcript-2 agonists.

### BACKGROUND

The transmembrane receptor TREM-like transcript 2 (TLT2) is one of four conserved triggering receptors expressed on myeloid cells (TREM) locus members, including TREM-I, TREM-2, and TLT-1 that are expressed in mice and humans (Sharif & Knapp (2008) Immunobiology 213: 701-713). Receptors encoded within the TREM locus have been shown to play an important role in modulating various aspects of the innate immune response (Sharif & Knapp (2008). Immunobiology 213: 701-713; Ford & McVicar (2009) Curr. Opin. Immunol. 21: 38-46; Netea et al., (2006) J. Leukoc. Biol. 80: 1454-1461). TLT2 is expressed on B cells, neutrophils, and macrophages and is up-regulated in response to inflammatory stimuli in vivo (King et al., (2006) J. Immunol. 176: 6012-6021). Recent studies have demonstrated that ligation of TLT2 synergistically enhances the migratory response, degranulation, and the respiratory burst of neutrophils in response to agonists that signal via G-protein-coupled receptors (GPCR) (Halpert et al., (2011) J. Immunol. 187: 2346-2355). Importantly, TLT2 ligation was not observed to potentiate the neutrophil response to agonists that signal via other classes of receptors, including growth factor receptors, Fc receptors or TLRs, which is in contrast to TREM-I or TREM-2, both of which modulate the cellular response to TLR-mediated signals (Sharif & Knapp (2008) Immunobiology 213: 701-713; Netea et al., (2006) J. Leukoc. Biol. 80: 1454-14611; Hamerman et al., (2006) J. Immunol. 177: 2051-2055; Klesney-Tait et al., (2006) Nat. Immunol. 7: 1266-1273).

### SUMMARY

TREM-like transcript 2 (TLT2), is expressed on neutrophils, macrophages, and B lymphocytes in mouse and human. Expression of TLT2 is up-regulated on neutrophils and macrophages in response to inflammatory stimuli in vivo. TLT2 synergizes with agonists that bind to G-protein coupled receptors (GPCR) to potentiate the neutrophil antibacterial and chemotactic response. Administration of anti-TLT2 mAb was found to enhance the acute inflammatory response in vivo, which was associated with increased neutrophil recruitment to sites of inflammation. Moreover, TLT2 ligation in vivo was found to potentiate chemokine and growth factor production indicating that TLT2 can exert both neutrophil intrinsic and extrinsic effects. The administration of anti-TLT2 mAb alone to the lung or peritoneum promotes neutrophil recruitment to the respective location, as well as the rapid production of G-CSF, CXCL1 (KC) and CXCL2 (MIP-2). Ligation of TLT2 on resident macrophages induced the production of G-CSF, CXCL1 and CXCL2 ex vivo, independent of secondary stimuli, demonstrating that TLT2 exerts a complementary functional effect on both neutrophils and macrophages. Thus, TLT2 activates a potent feed-forward loop wherein its ligation mediates the production of chemokines that recruit neutrophils to sites of infection/inflammation, while potentiating the response of these cells to chemokines and bacterial products that signal via GPCRs.

One aspect of the disclosure, therefore, encompasses embodiments of a method for enhancing an innate immune response of an animal or human subject, the method comprising the steps of delivering to a cell or population of cells an effective amount of an agent that specifically interacts with a TREM-like transcript 2 (TLT2) transmembrane receptor of the cell or population of cells and potentiates neutrophil and/or macrophage activation and/or migration.

In embodiments of this aspect of the disclosure, the cell or population of cells can be a neutrophil or population of neutrophils, wherein said cell or population of cells are isolated from an animal or human subject, a cultured neutrophil or population of neutrophils; or a neutrophil or population of neutrophils in an animal or human subject.

In embodiments of this aspect of the disclosure, the cell or population of cells can be a macrophage or population of macrophages, wherein said cell or population of cells are isolated from an animal or human subject, a cultured macrophage or population of macrophages; or a macrophage or population of macrophages in an animal or human subject.

In embodiments of this aspect of the disclosure, the agent can be an antibody, or a fragment thereof, capable of specifically binding to an extracellular domain of TLT2 of a neutrophil or a macrophage.

In embodiments of this aspect of the disclosure, the antibody can be a monoclonal antibody or a fragment thereof.

In embodiments of this aspect of the disclosure, the agent delivered to the cell or population of cells can initiate ligation of the TLT2 thereon.

In embodiments of this aspect of the disclosure, the agent delivered to the cell or population of cells can induce the release of at least one cytokine by the cell or population of cells, wherein the at least one cytokine can be characterized as capable of interacting with at least one G-protein coupled receptor of a neutrophil, thereby inducing neutrophil activation and/or migration.

In embodiments of this aspect of the disclosure, the agent delivered to the cell or population of cells can be mixed with a pharmaceutically acceptable carrier.

Another aspect of the disclosure encompasses embodiments of a method of potentiating a neutrophil response to a G-protein coupled receptor signaling comprising delivering to a neutrophil an effective amount of an agent specifically interacting with TREM-like transcript 2 (TLT2) transmembrane receptor of the neutrophil.

In embodiments of this aspect of the disclosure, the neutrophil can be an isolated neutrophil, a cultured neutrophil, or is a neutrophil of an animal or human subject.

Yet another aspect of the disclosure encompasses embodiments of a method of modulating an inflammatory response of an animal or human comprising delivering to the animal or human subject a composition comprising an effective amount of an agent specifically interacting with a ligand of a TREM-like transcript 2 (TLT2) transmembrane receptor of a neutrophil or a macrophage.

In embodiments of this aspect of the disclosure, the agent can be a recombinant polypeptide comprising a domain specifically binding to a TLT2-binding ligand.

In embodiments of this aspect of the disclosure, the domain specifically binding to a TLT2 agonist can be an extracellular region of TLT2, or a fragment thereof.

In embodiments of this aspect of the disclosure, the agent delivered to the animal or human subject can be combined with a pharmaceutically acceptable carrier.

In embodiments of this aspect of the disclosure, the method can further comprise delivering to the animal or human subject a heterogeneous nucleic acid expressing the recombinant polypeptide; and allowing expression of the recombinant polypeptide to be expressed in the animal or human subject, thereby delivering to the animal or human subject an effective amount of the agent specifically interacting with a ligand of a TREM-like transcript 2 (TLT2) transmembrane receptor of a neutrophil or a macrophage

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1F illustrate that the ligation of TLT2 in vivo potentiates the inflammatory response.

FIG. 1A illustrates the quantification of neutrophil infiltration into the ear in response to croton oil exposure. At the time points indicated following croton oil treatment, the number of infiltrating neutrophils in 4 mm biopsies was determined by assaying MPO activity.

FIG. 1B illustrates the weight of the 4 mm biopsy samples from ears of mice determined 4 h following the indicated treatment.

FIG. 1C shows that 12 h following the application of croton oil, histological sections were prepared and stained with hematoxylin and eosin. Scale bar in the lower right of each image represents 100 μm.

FIGS. 1D and 1E illustrate the analysis of soluble factor production in response to TLT2 ligation in vivo. The concentrations of soluble mediators in ear biopsies (FIG. 1D), or serum (FIG. 1E) in response to anti-TLT2 mAb treatment are shown as the mean±SEM of compiled experiments consisting of at least three mice per group.

FIG. 1F shows the concentration of soluble mediators measured in ear homogenates from mice administered anti-TLT2 mAb or PBS 4 h after croton oil treatment. The average concentration for 32 analytes was calculated from three animals per group. The fold difference in soluble factor concentration for anti-TLT2 mAb treated versus PBS control mice is depicted.

FIGS. 2A-2D illustrate that the in vivo administration of anti-TLT2 mAb induces recruitment of neutrophils to the lung and peritoneum. Data were compiled from multiple experiments and represent at least three animals per condition with the mean±SEM shown. Asterisks indicate significant differences in the number of leukocytes recruited compared to time 0 (FIGS. 2A and 2C), or a significant difference in the number of leukocytes recruited when comparing IC antibody-treated and anti-TLT2 monoclonal antibody-treated mice (FIGS. 2B and 2D).

FIG. 2A illustrates the number of leukocytes recruited to the lung in response to i.t. administration of 50 μg of anti-TLT2 mAb (1H4 F(ab′)₂) over time.

FIG. 2B shows the absolute number of neutrophils present in BAL at the indicated times after i.t. injection of either 1H4 F(ab′)₂ mAb or IC Ab as determined by flow cytometry.

FIG. 2C shows the number of leukocytes isolated from the peritoneum of mice over time following i.p. injection of 50 μg of anti-TLT2 mAb (1H4 F(ab′)₂).

FIG. 2D illustrates the measurement of neutrophil recruitment to the peritoneum in response to anti-TLT2 mAb or IC Ab.

FIGS. 3A-3E illustrate that ligation of TLT2 in vivo results in the production of neutrophil chemoattractants and growth factors.

FIG. 3A shows the maximum concentration of the indicated factors present in the lung following administration of anti-TLT2 mAb or IC Ab determined by Bio-Plex.

FIG. 3B shows that administration of anti-TLT2 mAb induces the production of soluble factors in the lung in a dose-dependent manner.

FIG. 3C illustrates the analysis of the kinetics of soluble factor (i.e. G-CSF, CXCL2, CXCL1, and IL-6) production in the lung following i.t. administration of either 50 μg of anti-TLT2 mAb (1H4 F(ab′)₂, solid square) or IC Ab (open square). Data are compiled from multiple experiments and represent at least three animals per condition with the means±SEM shown.

FIG. 3D illustrates analysis of soluble factor production in the lung of anti-TLT2 mAb-treated versus IC-treated mice. The maximum concentration for each of 32 analytes was determined in groups of 3 mice after receiving an i.t. injection of either anti-TLT2 mAb or IC Ab. The fold difference in soluble factor concentration for anti-TLT2 mAb-treated versus IC-treated mice is depicted.

FIG. 3E illustrates an analysis of soluble factor production in the peritoneum of anti-TLT2 mAb treated versus IC-treated mice. The maximum concentration for each of 32 analytes was determined in groups of 3 mice after receiving an i. t. injection of either anti-TLT2 mAb or IC Ab. The fold difference in soluble factor concentration for anti-TLT2 mAb treated versus IC mice is depicted.

FIGS. 4A-4C illustrate that TLT2 ligation on resident macrophages results in the production of soluble factors ex vivo.

FIG. 4A shows the concentration of the indicated factors present in the supernatant of primary resident peritoneal macrophages cultured 4 h in the presence of anti-TLT2 mAb or IC Ab.

FIG. 4B shows the analysis of the fold increase in production of soluble factors in supernatants of resident peritoneal macrophages treated with anti-TLT2 mAb compared to those treated with IC Ab over time.

FIG. 4C shows an analysis of activation marker (CD86. CD80, and CD69) expression on resident peritoneal macrophages after no treatment (filled), or 18 h following the i.p. administration of 50 μg of LPS (black line), or 50 μg anti-TLT2 mAb (grey line).

FIGS. 5A-5C illustrate that TLT2 ligation potentiates ROS production by murine neutrophils in response to FMLP. For FIG. 5A, the maximum ROS production for pretreated samples was normalized to controls that received FMLP alone. For FIG. 5C, the data were normalized to samples that received either FMLP or GM-CSF alone. The absolute values for ROS-dependent chemiluminescence are shown as well. All data represent the average of triplicate samples with mean±standard deviation shown and are representative of at least three independent experiments. Asterisks denote significance of values for αTLT2 mAb-treated samples compared to the respective control sample.

FIG. 5A shows the treatment with distinct αTLT2 mAbs (1H4 or 1C5) enhances the production of ROS in a dose-dependent manner following the addition of FMLP. Neutrophils (1×10⁶) were pre-incubated for 45 min with αTLT2 or isotype control (IC) mAb at the indicated concentrations, and then stimulated with FMLP (1 μM).

FIG. 5B shows the results from purified neutrophils incubated in the presence of αTLT2 mAb or LPS for either 15 min (upper panel) 45 min (middle panel), or 2 h (lower panel), then stimulated with 1 μM FMLP. Alternatively, neutrophils were pre-incubated for 15, 45 or 120 min with biotinylated 1H4 mAb (1 μg) that had been pre-mixed with streptavidin (SA) (20 μg) followed by addition of FMLP. ROS production was monitored at the indicated times.

FIG. 5C show TLT2 ligation does not alter ROS generation in response to GM-CSF. Purified neutrophils were incubated in the presence of αTLT2 or isotype control mAb for 45 min followed by stimulation with either FMLP (1 μM) or GM-CSF (1 ng/ml).

FIGS. 6A and 6B show that TLT2 ligation potentiates ROS production by neutrophils in response to FMLP, but not GM-CSF. Neutrophils (1×10⁶ per sample) were pretreated with isotype control or α-TLT2 mAb (100 ng/mL) for 45 min. The cells were then triggered by the addition of FMLP (FIG. 6A) or GM-CSF (FIG. 6B) and the maximum ROS-dependent chemiluminescence was measured. The data represent the average of triplicate samples with the mean±SD shown.

FIGS. 7A-7E illustrate that TLT2 ligation enhances the chemotactic response of neutrophils to FMLP.

FIGS. 8A-8I illustrate that TLT2 ligation potentiates neutrophil degranulation and up-regulation of the activation marker CD11b in response to GPCR-mediated signaling. At the indicated time points, the cells were harvested and stained to detect surface expression of CD11b and analyzed by flow cytometry. Filled histograms represent CD11b expression on neutrophils incubated in medium alone. Representative histograms of CD11b expression are shown. All data are representative of at least three independent experiments.

FIGS. 9A and 9B illustrate that TLT2 ligation does not increase FcR-mediated phagocytosis. Purified neutrophils (1×10⁶) were incubated either without stimulus, or with 100 ng/ml of the intact or F(ab′)₂ fragment of 1H4, 1 μg/ml biotinylated 1C5 plus 20 μg/ml streptavidin, or 2 μg/ml of LPS for 10 min. The cells were washed and 5×10⁶ FITC-conjugated 2 μm polystyrene beads were added. The opsonized beads were pre-incubated with mouse polyclonal αFITC. Following a 15 min incubation, the cells were harvested, extracellular FITC fluorescence was quenched with trypan blue and the cells were analyzed by flow cytometry to detect phagocytosis of FITC labeled particles. The data in FIG. 9A are representative of 3 independent experiments, and the mean values for fold increase in phagocytosis±SD in FIG. 9B were calculated from the three independent experiments.

FIG. 9A shows representative histograms.

FIG. 9B shows the mean fold increase in the percentage of cells containing phagocytosed beads compared to cells incubated in medium alone with non-opsonized beads.

FIGS. 10A and 10B illustrate TLT2 ligation results in enhanced migration of neutrophils in response to a variety of chemoattractants in vitro.

FIGS. 11A-11C illustrate the administration of αTLT2 mAb results in enhanced accumulation of neutrophils at sites of inflammation in vivo.

FIGS. 12A-12C illustrate TLT2 ligation enhances neutrophil recruitment into sites of inflammation in vivo.

FIG. 12A shows purified neutrophils from CD45.1 mice labeled with either CFSE, CMPTX, or left unlabeled.

FIG. 12B shows ligation of TLT2 increases the relative frequency of neutrophils recruited into the lungs of recipient mice.

FIG. 12C shows ligation of TLT2 increases the absolute number of neutrophils recruited into lungs of recipient mice.

FIG. 13 illustrates anti-TLT2 mAb-mediated protection against challenge with S. pneumonia.

FIG. 14 illustrates the effects of the intravenous administration of soluble TLT2:Fc fusion protein blocks neutrophil recruitment to the lung in response to intratracheal injection of LPS

FIG. 15 illustrates the amino acid sequence (SEQ ID No.: 1) of a fusion protein consisting of a TLT2 extracellular region and a human IgG1 constant region.

The details of some exemplary embodiments of the methods and systems of the present disclosure are set forth in the description below. Other features, objects, and advantages of the disclosure will be apparent to one of skill in the art upon examination of the following description, drawings, examples and embodiments. It is intended that all such additional systems, methods, features, and advantages included within this description be within the scope of the present disclosure.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates, which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended embodiments, the singular forms “a,” “an,” and “the” include plural referents unless the context clearly dictates otherwise. Thus, for example, reference to “a support” includes a plurality of supports. In this specification and in the embodiments that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure, refer to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods compared to those of the corresponding compositions or methods disclosed herein.

Prior to describing the various embodiments, the following definitions are provided and should be used unless otherwise indicated.

### DEFINITIONS

In describing the disclosed subject matter, the following terminology will be used in accordance with the definitions set forth below.

The terms “administering” and “administration” as used herein refer to introducing a composition of the present disclosure into a subject. The preferred route of administration of the composition is, but not limited to, intravenous.

The term “specific binding” as used herein refers to the specific recognition of one molecule, of two different molecules, compared to substantially less recognition of other molecules. Generally, the molecules have areas on their surfaces or in cavities giving rise to specific recognition between the two molecules. Exemplary of specific binding are antibody-antigen interactions, enzyme-substrate interactions, polynucleotide interactions, and so forth.

The term “antibody” as used herein refers to an immunoglobulin that specifically binds to, and is thereby defined as complementary with, a particular spatial and polar organization of another molecule. The antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, IgY, etc. Fragments thereof may include Fab, Fv and F(ab′)₂, Fab′, scFv, and the like. In addition, aggregates, polymers, and conjugates of immunoglobulins or their fragments can be used where appropriate so long as binding affinity for a particular molecule is maintained.

Methods of making polyclonal and monoclonal antibodies are known in the art. Polyclonal antibodies are generated by immunizing a suitable animal, such as a mouse, rat, rabbit, sheep or goat, with an antigen of interest. To enhance immunogenicity, the antigen can be linked to a carrier prior to immunization. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Furthermore, the antigen may be conjugated to a bacterial toxoid, such as toxoid from diphtheria, tetanus, cholera, etc., to enhance the immunogenicity thereof. Rabbits, sheep, mice, rats, hamsters, horses, cows and goats are often used for the preparation of polyclonal sera when large volumes of sera are desired. These animals are good design choices also because of the availability of labeled anti-host antibodies. Immunization is performed by mixing or emulsifying the antigen in saline, preferably in an adjuvant such as Complete Freund's Adjuvant (“CFA”) and injected. The animal is boosted 2-6 weeks later with one or more injections of the antigen in saline, often with the antigen emulsified with Incomplete Freund's adjuvant (“IFA”). Antibodies may also be generated by in vitro immunization, using methods known in the art. Polyclonal antisera is then obtained from the immunized animal.

As used herein, the terms “antigen-binding site” or “binding portion” refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}.

As used herein, the term “Fab′,” refers to a polypeptide that is a heterodimer of the variable domain and the first constant domain of an antibody heavy chain, plus the variable domain and constant domain of an antibody light chain, plus at least one additional amino acid residue at the carboxy terminus of the heavy chain C_{H1} domain including one or more cysteine residues. F(ab′)₂ antibody fragments are pairs of Fab′ antibody fragments which are linked by a covalent bond(s). The Fab′ heavy chain may include a hinge region. This may be any desired hinge amino acid sequence. Alternatively the hinge may be entirely omitted in favor of a single cysteine residue or, a short (about 1-10 residues) cysteine-containing polypeptide. In certain applications, a common naturally occurring antibody hinge sequence (cysteine followed by two prolines and then another cysteine) is used; this sequence is found in the hinge of human IgG1 molecules (E. A. Kabat, et al., Sequences of Proteins of Immunological Interest 3rd edition (National Institutes of Health, Bethesda, Md., 1987)). In other embodiments, the hinge region is selected from another desired antibody class or isotype.

As used herein, the term “hinge region” refers to an amino acid sequence located between C_{H1} and C_{H2} in native immunoglobulins or any sequence variant thereof. Analogous regions of other immunoglobulins will be employed, although it will be understood that the size and sequence of the hinge region may vary widely. For example, the hinge region of a human IgG.sub.1 is only about 10 residues, whereas that of human IgG3 is about 60 residues.

As used herein, the term Fv refers to a covalently or non-covalently-associated heavy and light chain heterodimer which does not contain constant domains. As used herein, the terms “Fv-SH” or “Fab′-SH” refers to an Fv or Fab′ polypeptide having a cysteinyl free thiol. The free thiol is in the hinge region, with the light and heavy chain cysteine residues that ordinarily participate in inter-chain bonding being present in their native form. In the most preferred embodiments of this invention, the Fab′-SH polypeptide composition is free of heterogeneous proteolytic degradation fragments and is substantially (greater than about 90 mole percent) free of Fab′ fragments wherein heavy and light chains have been reduced or otherwise derivatized so as not to be present in their native state, e.g. by the formation of aberrant disulfides or sulfhydryl addition products

The term “region” as used herein refers interchangeably with “domain” and refers to a functional unit of a peptide or polypeptide sequence.

As used herein, the term “engineered protein” refers to a non-naturally-occurring polypeptide. The term encompasses, for example, a polypeptide that comprises one or more changes, including additions, deletions or substitutions, relative to a naturally occurring polypeptide, wherein such changes were introduced by recombinant DNA techniques. The term also encompasses a polypeptide that comprises an amino acid sequence generated by man, an artificial protein, a fusion protein, and a chimeric polypeptide. Once expressed, recombinant peptides, polypeptides and proteins can be purified according to standard procedures known to one of ordinary skill in the art, including ammonium sulfate precipitation, affinity columns, column chromatography, gel electrophoresis and the like. Substantially pure compositions of about 50 to 99% homogeneity are preferred, and 80 to 95% or greater homogeneity are most preferred for use as therapeutic agents. Engineered proteins may be produced by any means, including, for example, peptide, polypeptide, or protein synthesis.

For the purposes of this disclosure, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material includes material in native and recombinant form.

A pharmaceutical composition according to the disclosure may include, but is not limited to, an immunotherapeutic composition comprising an anti-TLT2 antibody, or a TLT2-binding fragment thereof. An immunotherapeutic composition includes a vaccine. Suitably, the pharmaceutical composition may further include or be mixed with a pharmaceutically-acceptable carrier. By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water.

The term “nucleic acid” as used herein refers to any natural and synthetic linear and sequential arrays of nucleotides and nucleosides, for example cDNA, genomic DNA, mRNA, tRNA, oligonucleotides, oligonucleosides and derivatives thereof. For ease of discussion, such nucleic acids may be collectively referred to herein as “constructs,” “plasmids,” or “vectors.” Representative examples of the nucleic acids of the present disclosure include bacterial plasmid vectors including expression, cloning, cosmid and transformation vectors such as, but not limited to, pBR322, animal viral vectors such as, but not limited to, modified adenovirus, influenza virus, polio virus, pox virus, retrovirus, insect viruses (baculovirus), and the like, vectors derived from bacteriophage nucleic acid, and synthetic oligonucleotides like chemically synthesized DNA or RNA. The term “nucleic acid” further includes modified or derivatized nucleotides and nucleosides such as, but not limited to, halogenated nucleotides such as, but not only, 5-bromouracil, and derivatized nucleotides such as biotin-labeled nucleotides.

The terms “polypeptide” and “protein” as used herein refer to a polymer of amino acids of three or more amino acids in a serial array, linked through peptide bonds. The term “polypeptide” includes proteins, protein fragments, protein analogues, oligopeptides and the like. The term “polypeptides” contemplates polypeptides as defined above that are encoded by nucleic acids, produced through recombinant technology (isolated from an appropriate source such as a bird), or synthesized. The term “polypeptides” further contemplates polypeptides as defined above that include chemically modified amino acids or amino acids covalently or non-covalently linked to labeling ligands. Polypeptides that are not naturally part of a particular organism's protein, polypeptide or peptide complement are referred to as “foreign polypeptides,” “heterologous polypeptide” or “exogenous polypeptide.”

The term “fragment” as used herein may also refer to an isolated portion of a polypeptide, wherein the portion of the polypeptide is cleaved from a naturally occurring polypeptide by proteolytic cleavage by at least one protease, or is a portion of the naturally occurring polypeptide synthesized by chemical methods well known to one of skill in the art.

The terms “gene” or “genes” as used herein refer to nucleic acid sequences (including both RNA and DNA) that encode genetic information for the synthesis of a whole RNA, a whole protein, or any portion of such whole RNA or whole protein. Genes that are not naturally part of a particular organism's genome are referred to as “foreign genes,” “heterologous genes” or “exogenous genes” and genes that are naturally a part of a particular organism's genome are referred to as “endogenous genes.” The term “gene product” refers to RNAs or proteins that are encoded by the gene. “Foreign gene products” are RNA or proteins encoded by “foreign genes” and “endogenous gene products” are RNA or proteins encoded by endogenous genes. “Heterologous gene products” are RNAs or proteins encoded by “foreign, heterologous or exogenous genes” and are, therefore, not naturally expressed in the cell.

The terms “expressed” or “expression” as used herein refer to the transcription from a gene to give an RNA nucleic acid molecule at least complementary in part to a region of one of the two nucleic acid strands of the gene. The terms “expressed” or “expression” as used herein also refer to the translation from said RNA nucleic acid molecule to give a protein, a polypeptide, or a portion or fragment thereof.

The term “operably linked” refers to an arrangement of elements wherein the components so described are configured so as to perform their usual function. Control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The terms “transcription regulatory sequences” and “gene expression control regions” as used herein refer to nucleotide sequences that are associated with a gene nucleic acid sequence and which regulate the transcriptional expression of the gene. Exemplary transcription regulatory sequences include enhancer elements, hormone response elements, steroid response elements, negative regulatory elements, and the like. The “transcription regulatory sequences” may be isolated and incorporated into a vector nucleic acid to enable regulated transcription in appropriate cells of portions of the vector DNA. The “transcription regulatory sequence” may precede, but is not limited to, the region of a nucleic acid sequence that is in the region 5′ of the end of a protein coding sequence that may be transcribed into mRNA. Transcriptional regulatory sequences may also be located within a protein coding region, in regions of a gene that are identified as “intron” regions, or may be in regions of nucleic acid sequence that are in the region of nucleic acid.

The term “expression vector” as used herein refers to a nucleic acid vector that comprises a gene expression control region operably linked to a nucleotide sequence coding at least one polypeptide. As used herein, the term “regulatory sequences” includes promoters, enhancers, and other elements that may control gene expression. Standard molecular biology textbooks (for example, Sambrook et al., eds., 1989, “Molecular Cloning: A Laboratory Manual,” 2nd ed., Cold Spring Harbor Press) may be consulted to design suitable expression vectors that may further include an origin of replication and selectable gene markers. It should be recognized, however, that the choice of a suitable expression vector and the combination of functional elements therein depends upon multiple factors including the choice of the host cell to be transformed and/or the type of protein to be expressed.

The terms “recombinant nucleic acid” and “recombinant DNA” as used herein refer to combinations of at least two nucleic acid sequences that are not naturally found in a eukaryotic or prokaryotic cell. The nucleic acid sequences include, but are not limited to, nucleic acid vectors, gene expression regulatory elements, origins of replication, suitable gene sequences that when expressed confer antibiotic resistance, protein-encoding sequences, and the like. The term “recombinant polypeptide” is meant to include a polypeptide produced by recombinant DNA techniques such that it is distinct from a naturally occurring polypeptide either in its location, purity or structure. Generally, such a recombinant polypeptide will be present in a cell in an amount different from that normally observed in nature.

The techniques used to isolate and characterize the nucleic acids and proteins of the present disclosure are well known to those of skill in the art, and standard molecular biology and biochemical manuals may be consulted to select suitable protocols without undue experimentation (see, for example, Sambrook et al., “Molecular Cloning: A Laboratory Manual,” 2nd ed., 1989, Cold Spring Harbor Press; the content of which is incorporated herein by reference in its entirety).

A “cyclic polymerase-mediated reaction” refers to a biochemical reaction in which a template molecule or a population of template molecules is periodically and repeatedly copied to create a complementary template molecule or complementary template molecules, thereby increasing the number of the template molecules over time.

“Denaturation” of a template molecule refers to the unfolding or other alteration of the structure of a template so as to make the template accessible to duplication. In the case of DNA, “denaturation” refers to the separation of the two complementary strands of the double helix, thereby creating two complementary, single stranded template molecules. “Denaturation” can be accomplished in any of a variety of ways, including by heat or by treatment of the DNA with a base or other denaturant.

“DNA amplification” as used herein refers to any process that increases the number of copies of a specific DNA sequence by enzymatically amplifying the nucleic acid sequence. A variety of processes are known. One of the most commonly used is the polymerase chain reaction (PCR), which is defined and described in later sections below. The PCR process of Mullis is described in U.S. Pat. Nos. 4,683,195 and 4,683,202. PCR involves the use of a thermostable DNA polymerase, known sequences as primers, and heating cycles that separate the replicating deoxyribonucleic acid (DNA) strands and exponentially amplify a gene of interest. Any type of PCR, such as quantitative PCR, RT-PCR, hot start PCR, LAPCR, multiplex PCR, touchdown PCR, etc., may be used. Advantageously, real-time PCR is used. In general, the PCR amplification process involves an enzymatic chain reaction for preparing exponential quantities of a specific nucleic acid sequence. It requires a small amount of a sequence to initiate the chain reaction and oligonucleotide primers that will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed.

As used herein, the term “protein” refers to a large molecule composed of one or more chains of amino acids in a specific order. The order is determined by the base sequence of nucleotides in the gene coding for the protein. Proteins are required for the structure, function, and regulation of the body's cells, tissues, and organs. Each protein has a unique function.

### Abbreviations:

BAL. Bronchalveloar lavage; GPCR, G protein-coupled receptor; IC, isotype control; i.t., intratracheal; MPO, myeloperoxidase; PRR, pattern recognition receptor; ROS, reactive oxygen species; SA, streptavidin; TLT2, TREM-like transcript 2; TMB, tetramethylbenzidine; TREM, triggering receptor expressed on myeloid cells.

### DESCRIPTION

The present disclosure encompasses compositions and methods of use, therefore, for the modulation of the innate immune system, and in particular the activation and migration of neutrophils and macrophages that can respond to infectious agents in an animal or human subject. In particular, the present disclosure describes the use of pharmaceutically acceptable compositions comprising one or more species of monoclonal antibodies that are specifically directed to the extracellular domain of TLT2 (TREM-like transcript 2). On contact with antigenic moieties of infectious particles such as, but not limited to, bacteria, viruses and the like, there is an up-regulation of TLT2, enhanced generation of ROS and the generation of cytokines by neutrophils or macrophages. Most advantageously, the ligation of the TLT2 on neutrophils leads to an enhanced migration of the cells to a site of infection and an enhanced inflammatory response. In addition, the present disclosure describes the use of agents, and in particular of antibodies directed to the extracellular domain of TLT2 on the surface of macrophages. This leads both to an increase in macrophage production of neutrophil-stimulating cytokines that further expand the capacity of the latter to migrate to the infectious site. Accordingly, the use of TLT2-specific antibodies such as, but not limited to, monoclonal antibodies as a method of enhancing the innate immune system in response to stimuli such as an infectious agent, is disclosed

Administration of an anti-TLT2 monoclonal antibody in vivo has been shown to enhance neutrophil recruitment to local sites of inflammation (Halpert et al., (2011) J. Immunol. 187: 2346-2355). This was shown to be due to the direct action of TLT2 on neutrophils resulting in their enhanced responsiveness to chemoattractants produced in association with inflammation (Halpert et al., (2011) J. Immunol. 187: 2346-2355). However, because TLT2 is expressed on other cell types it is likely that administration of anti-TLT2 mAb in vivo may influence the inflammatory response via other mechanisms. It has now been shown that administration of anti-TLT2 mAb in conjunction with an acute inflammatory stimulus (i.e. croton oil) resulted in enhanced tissue damage that is associated with increased neutrophil recruitment. It was also shown that TLT2 ligation in conjunction with administration of croton oil leads to the increased production of growth factors (G-CSF) and chemokines (CXCLI and CXCL2) that act on neutrophils. It is contemplated that any monoclonal antibody directed specifically to an antigenic region of the extracellular domain of the TLT2 may be suitable for use in embodiments of the methods of the present disclosure. Particularly advantageous, but not limiting, are the monoclonal antibodies 1H4 and 1C5 as disclosed by Halpert et al., (2011) J. Immunol. 187: 2346-2355, and incorporated herein by reference in its entirety.

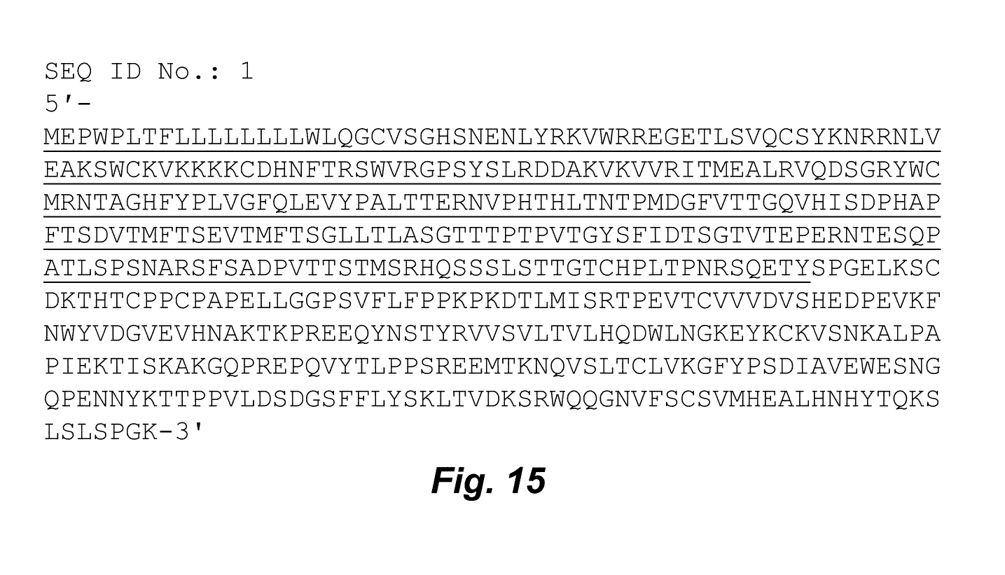

The disclosure, therefore, further provides for methods of modulating, and in particular, of reducing an inflammatory response by an animal or human subject by delivering to the subject a pharmaceutically acceptable composition that includes an agent that can reduce the availability of a ligand (i.e. an agonist) of the TLT2, thereby acting in reverse of the effect of an anti-TLT2 antibody. It is contemplated, for example, but not intended to be limiting, that said agent may be a fusion protein or polypeptide that combines an isolated extracellular domain of a TLT2 protein conjugated to the Fc-region of an immunoglobulin. An example of such a fusion protein construct having the amino acid sequence SEQ ID No.: 1 is illustrated in FIG. 15. Delivery or generation of this fusion polypeptide to the circulatory system of an animal or human subject can allow the TLT2 domain of the fusion product to selectively bind to an endogenous TLT2 ligand, thereby reducing the effective concentration thereof and its ability to interact with and bind to the TLT2 protein on the surface of the neutrophils and/or macrophages that have invaded the site of an infection or inflammation. Accordingly, there can be a reduction in the inflammation. While it is contemplated that a useful anti-inflammatory composition of the disclosure is a fusion protein as described in King et al., (2006) J. Immunol. 176: 6012 incorporated herein by reference in its entirety, any agent that specifically binds to a TLT2 ligand may be used in the embodiments of the disclosure. While it is contemplated that a TLT2 agonist-binding construct may be prepared and delivered to an animal or human subject, it is further within the scope of the disclosure for a heterogeneous nucleic acid encoding such a fusion protein of the disclosure and operably linked advantageously to a gene expression-controlling element and optionally inserted in a genetic vector to be delivered to the subject animal or human. Once in the recipient animal or human, the heterogenous nucleic acid may be expressed to deliver the TLT2 agonist-binding polypeptide to the circulatory system and hence to a site of inflammation.

### TLT2 Ligation Potentiates the Functional Response of Neutrophils to FMLP:

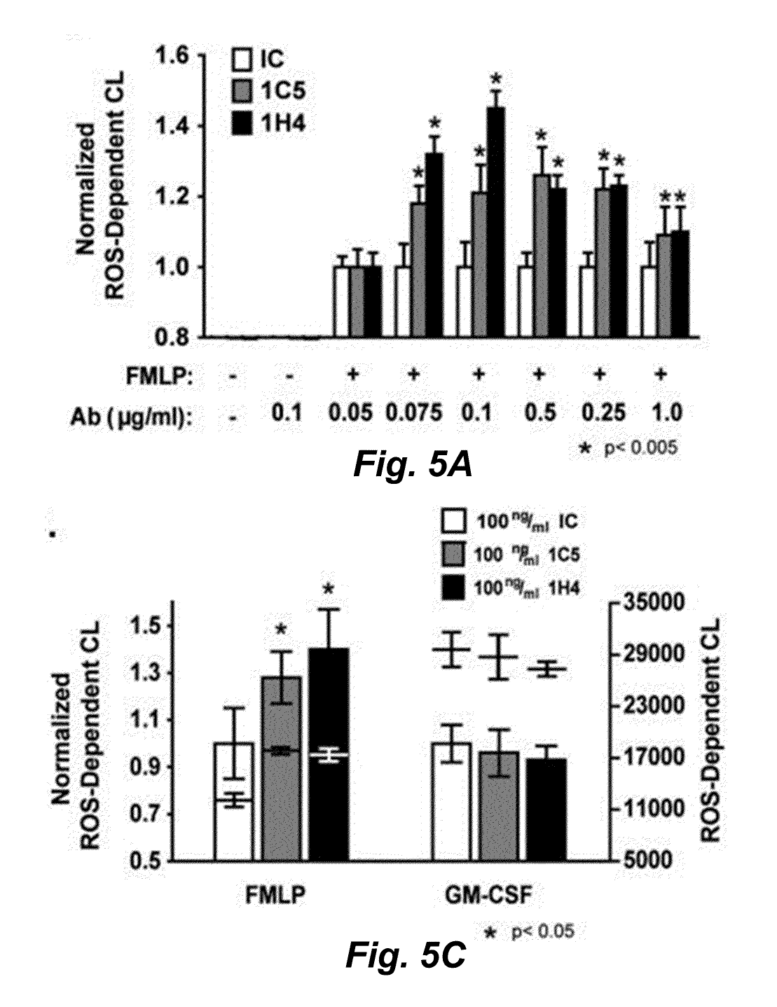

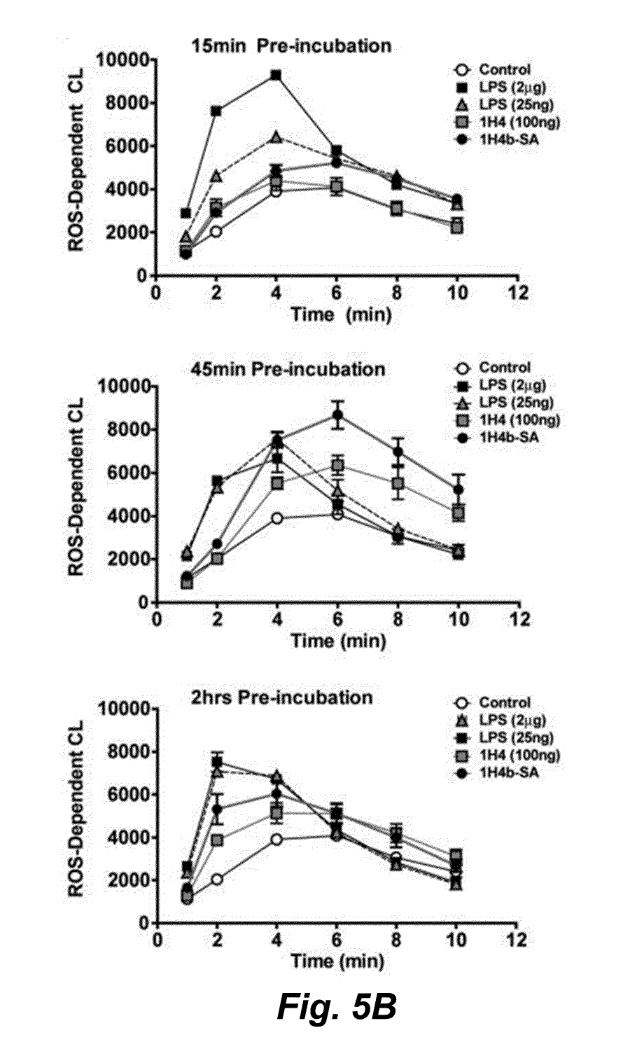

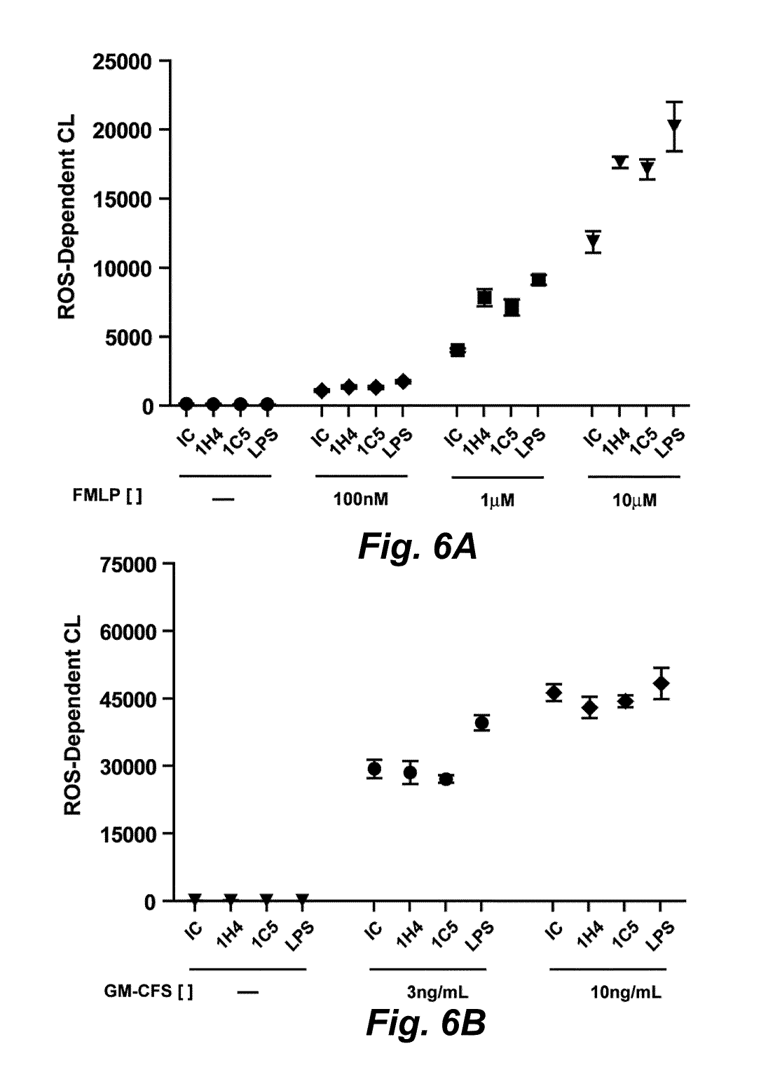

It has been demonstrated that TLT2 is constitutively expressed by both macrophages and neutrophils (King et al., (2006) J. Immunol. 176: 6012). Under experimental inflammatory conditions, including administration of thioglycolate, LPS, or staphylococcal enterotoxin B in vivo, responding macrophages and neutrophils exhibit significant up-regulation of TLT2 on their surface (King et al., (2006) J. Immunol. 176: 6012). This rapid up-regulation of TLT2 by neutrophils indicates that TLT2 may play a role in regulation of neutrophil function at sites of inflammation. A principle function of neutrophils is to respond to bacterial pathogens; therefore experiments were performed to determine if ligation of TLT2 with mAb enhances the antimicrobial activity of neutrophils by inducing ROS generation. To determine the effect that engagement of TLT2 has on ROS production, neutrophils were isolated from murine bone marrow and were analyzed using a chemiluminescence assay to measure the generation of ROS. As seen in FIGS. 5A and 6, ligation of TLT2 with either of the mAbs 1H4 or 1C5 alone does not induce a respiratory burst response.

### TLT2 Ligation Potentiates ROS Production by Murine Neutrophils in Response to FMLP:

Neutrophils generate ROS in response to a variety of extracellular stimuli including inflammatory cytokines and growth factors, as well as bacterial products such as LPS and formylated peptides derived from prokaryotic pathogens. FMLP is a short, formylated bacterial peptide that is released during degradation of the bacterial membrane and is a strong chemoattractant for phagocytic cells such as neutrophils (Rabiet et al., (2007) Biochimie 89: 1089). The response of innate immune cells to formylated peptides is mediated by binding to the FMLP receptor, a GPCR, resulting in activation of phospholipase C, protein kinase C, and calcium mobilization (Hyduk et al., (2007) Blood. 109:176).

Experiments were performed to determine if pre-incubation of neutrophils with αTLT2 mAb exerted a priming effect resulting in a change in the kinetics or magnitude of the respiratory burst elicited in response to FMLP. Pre-incubation with αTLT2 mAb prior to stimulation with FMLP resulted in a substantial increase in ROS production compared to neutrophils incubated with FMLP alone (FIGS. 5A-5C and 6). In contrast, pre-incubation of neutrophils with isotype control mAb had no effect on ROS production (FIG. 6) or on the subsequent response following addition of FMLP, regardless of the concentration of isotype control antibody used (FIGS. 5A-5C).

The potentiation of ROS production occurs over a broad range of αTLT2 mAb concentrations (FIG. 5A). When the potentiation of FMLP-induced ROS generation induced by αTLT2 mAb pre-treatment is compared to that induced by pre-treatment with LPS, a prototypic priming agent, the effects, although similar in magnitude, differ in their kinetics (i.e. initiation and duration). The priming effect of LPS is very rapid, as seen in FIG. 5B (upper panel). After a 15 min pre-incubation with LPS, the kinetics of the response to FMLP are shifted, and the overall magnitude of the response is substantially increased, compared to treatment of cells with FMLP alone. In contrast, pre-incubation with αTLT2 mAb for 15 min fails to significantly alter ROS production induced by FMLP. However, after a 45 min pre-incubation with αTLT2 mAb, ROS production in response to FMLP is similar to that observed with LPS pre-treatment in terms of magnitude. Whereas cells pretreated with LPS exhibited an accelerated, transient potentiation of ROS production, TLT2 ligation did not accelerate the kinetics of the response, but was observed to potentiate ROS production for a prolonged period of time. Finally, pre-treatment of neutrophils for 2 h with αTLT2 mAb resulted in a slight shift in the kinetics of the response similar to that observed with LPS treatment, as well as a prolongation of the potentiated response (FIG. 5B).

Potentiation of ROS production can be further enhanced by secondary cross-linking as demonstrated by treatment of neutrophils with biotinylated 1C5 in the presence of streptavidin, which results in a further enhancement of ROS production in response to FMLP at all time points assayed (FIG. 5B). This observation indicates that cross-linking of TLT2 elicits an enhanced response to FMLP. In summary, whereas ligation of TLT2 alone does not induce the generation of ROS by neutrophils, cross-linking of this receptor serves to potentiate ROS production in response to FMLP. Although ROS generation was similar for neutrophils primed either with αTLT2 mAb or LPS in terms of magnitude, significant differences were observed in the kinetics of ROS production. TLT2 ligation primarily acts to enhance the magnitude of ROS production over an extended period of time, but does not alter the kinetics of the response. In contrast, LPS accelerates the kinetics and increases the magnitude of the response in a transient manner. These data show that the mechanism by which TLT2 ligation potentiates ROS production is different from that associated with the priming effect of LPS.

In addition to FMLP, other stimuli, including growth factors induce a respiratory burst response in neutrophils. Therefore, experiments were performed to determine if the significant enhancement in ROS generation induced by TLT2 ligation in response to FMLP would be observed in response to other agonists. The GM-CSF receptor (also known as CD116) is expressed on several cell types, including mature neutrophils. GM-CSF, like FMLP, has been shown to elicit ROS production by neutrophils; however the GM-CSF receptor mediates a phosphotyrosine-based signal leading to ROS production (Al-Shami et al., (1998) J. Biol. Chem. 1998; 273:1058). As seen in FIG. 5C, unlike the response to FMLP, ROS generation by neutrophils in response to GM-CSF was not potentiated by ligation of TLT2.

In addition to promoting the generation of ROS, FMLP is a potent chemotactic agent for neutrophils (Rabiet et al., (2007) Biochimie 89: 1089). Because ligation of TLT2 was observed to potentiate ROS production in response to FMLP, experiments were performed to determine if the chemotactic response of neutrophils to FMLP is also potentiated. Medium containing varied concentrations of FMLP receptor agonist were placed in the bottom chamber of a 3 μm transwell device and neutrophils were placed in the upper chamber. Anti-TLT2 or isotype control mAb, or medium alone was added to the neutrophils just prior their addition to the upper chamber and after a 1 h incubation at 37° C., cells that migrated to the lower chamber were collected and counted.

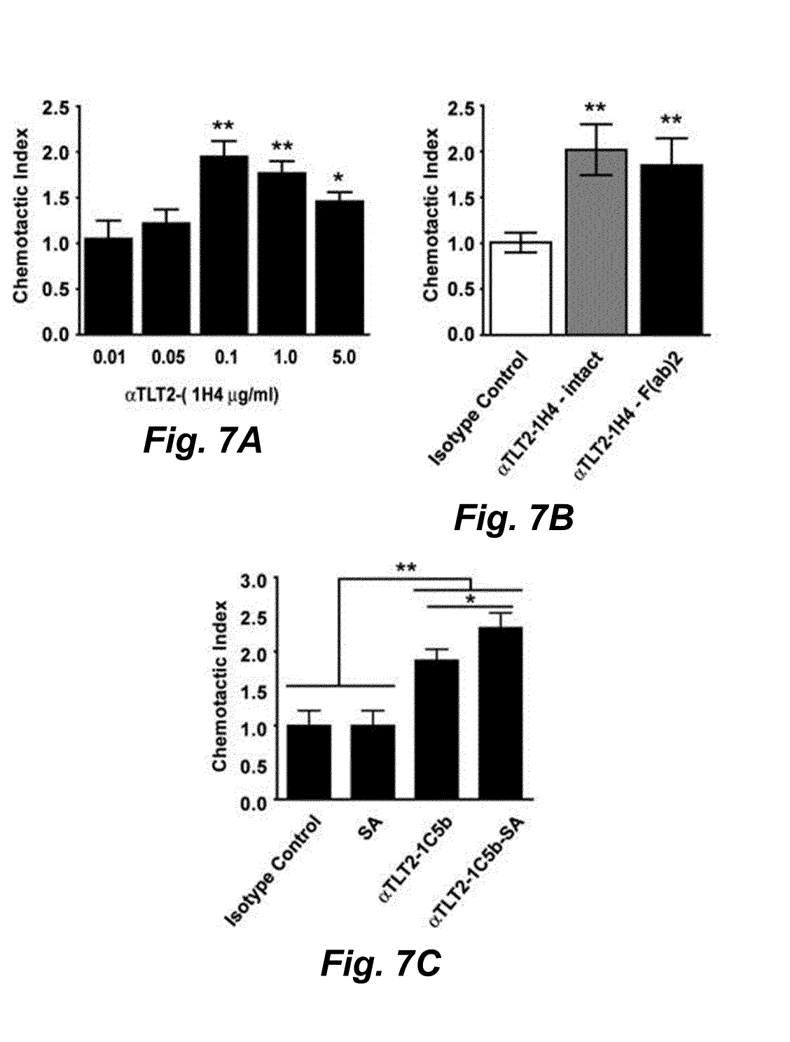

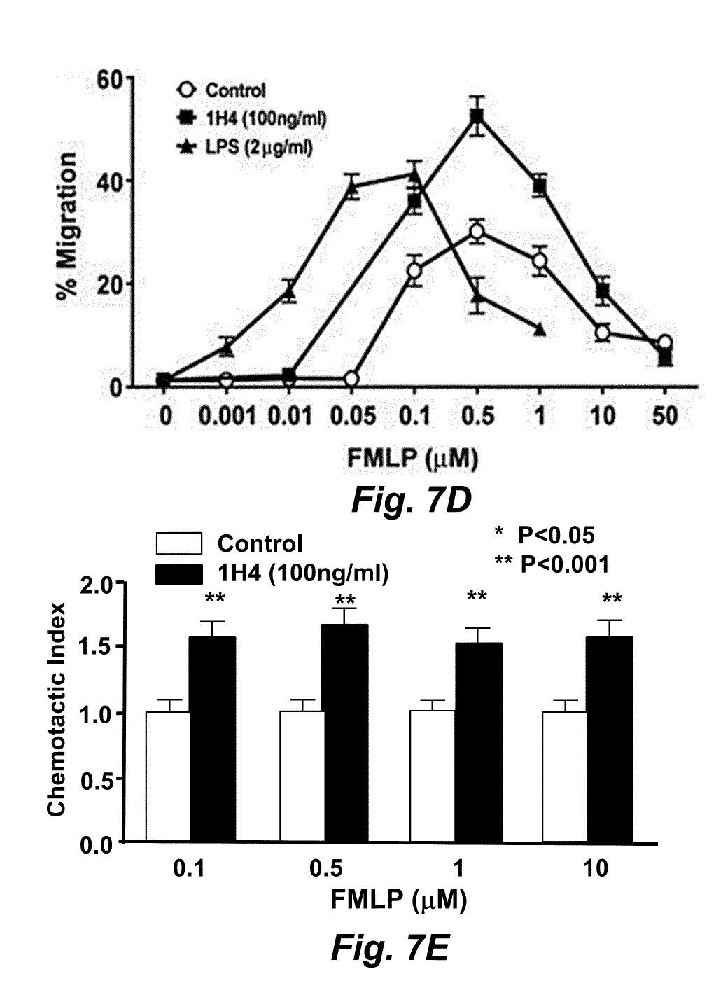

As shown in FIG. 7A, inclusion of αTLT2 mAb in these assays resulted in a substantial increase in the number of cells migrating through the transwell in response to FMLP. Of note, the concentration of αTLT2 mAb that maximally potentiates the chemotactic response of neutrophils is the same as that which provides maximal potentiation of ROS production. To rule out the possibility that Fc receptor-mediated signaling generated by the binding of intact antibodies to neutrophils was responsible for these observed effects, F(ab′)₂ fragments of the 1H4 mAb were generated and used in these assays (FIG. 7B). The observed potentiation of cell migration in response to FMLP was identical whether cells were treated with F(ab′)₂ fragments or intact 1H4, suggesting that signals delivered via TLT2 were responsible for the observed effects on ROS production and cell migration.

### TLT2 Ligation Enhances the Chemotactic Response of Neutrophils to FMLP:

Additional experiments were performed to determine if secondary cross-linking would further enhance this effect using biotinylated 1C5, either alone or in the presence of streptavidin. As seen in FIG. 7C, the inclusion of streptavidin alone has no effect on cell migration in response to FMLP, whereas the inclusion of biotinylated 1C5 mAb alone results in a nearly two-fold increase in the number of migrating cells. As seen in ROS assays, the inclusion of streptavidin and biotinylated 1C5 mAb results in a further enhancement, suggesting again that secondary cross-linking of TLT2 is responsible for this effect.

While not wishing to be bound by any one theory, a possible mechanism by which TLT2 ligation potentiates the response to FMLP is by decreasing the neutrophil threshold of sensitivity for agonist binding to the FMLP receptor. If this were true, then one would expect to see a shift in the dose response to FMLP in neutrophils on which TLT2 has been ligated. To determine if this is the case, neutrophil migration was assayed over a wide range of FMLP concentrations in the presence or absence of αTLT2 mAb. As seen in FIG. 7D, the inclusion of an optimal concentration of αTLT2 mAb potentiates neutrophil migration over a wide range of FMLP concentrations. However, TLT2 ligation does not result in a shift in the dose response to FMLP. Indeed, the observed potentiation in the number of migrating neutrophils treated with αTLT2 mAb is proportional regardless of the FMLP concentration as the ligation of TLT2 resulted in a chemotactic index (approximately 1.7) that is nearly identical for all concentrations of FMLP tested (FIG. 7E).

In contrast, inclusion of LPS shifts the chemotactic response curve to FMLP, suggesting that it lowers the threshold of sensitivity to this chemoattractant (FIG. 7D). Collectively, these results suggest a potential role for TLT2 in amplifying the cellular response to FMLP, as opposed to altering the threshold of sensitivity of the FMLP receptor. This suggests that TLT2 ligation could amplify the magnitude of the signal delivered via the FMLP receptor and/or prolong the duration of that signal thereby enhancing the cellular functional response. These data support the conclusion that the mechanism by which TLT2 modifies the response to GPCR-mediated signaling is distinct from that of LPS.

### Ligation of TLT2 Specifically Potentiates the Neutrophil Response to GPCR-Mediated Signaling:

Besides triggering both ROS production and neutrophil migration, FMLP and GM-CSF induce degranulation of murine neutrophils. The release of specific granules in response to these stimuli has been shown to mediate the rapid expression of the integrin CD11b resulting in an increase in cell adhesion and is thought to be a mechanism to promote transmigration of neutrophils into inflamed tissues (Borregaard N. Immunity 33:657). Because the rapid up-regulation of CD11b is associated with cellular activation, experiments were performed to determine if TLT2 ligation alone was sufficient to induce the up-regulation of CD11b, or if ligation of the TLT2 receptor potentiated up-regulation of CD11b induced by FMLP or the complement component C5a, which bind to GPCRs, versus GM-CSF or TLR agonists, which activate distinct signaling pathways that do not utilize heterotrimeric G-proteins.

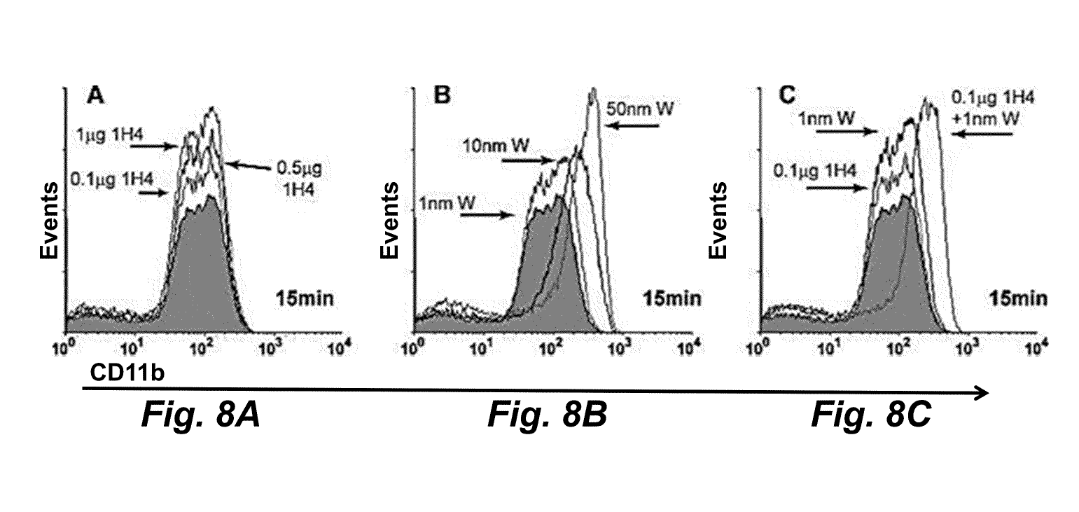

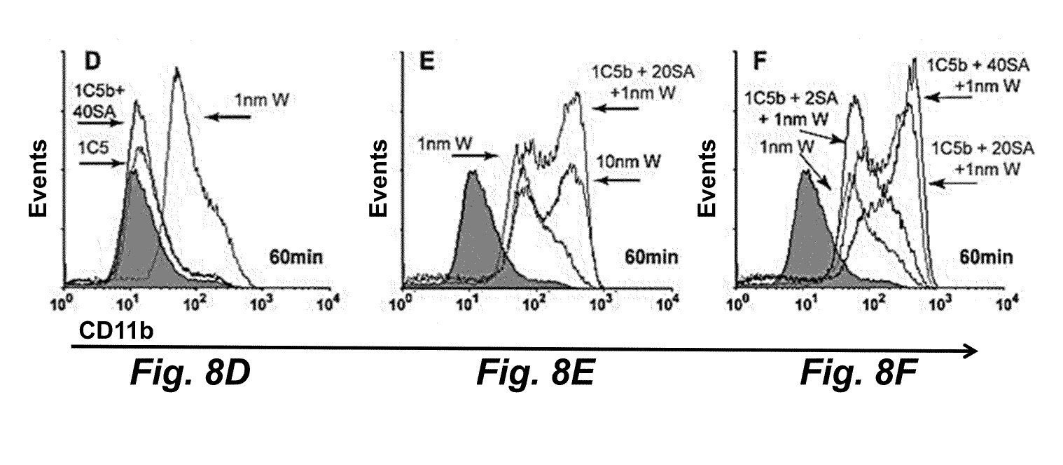

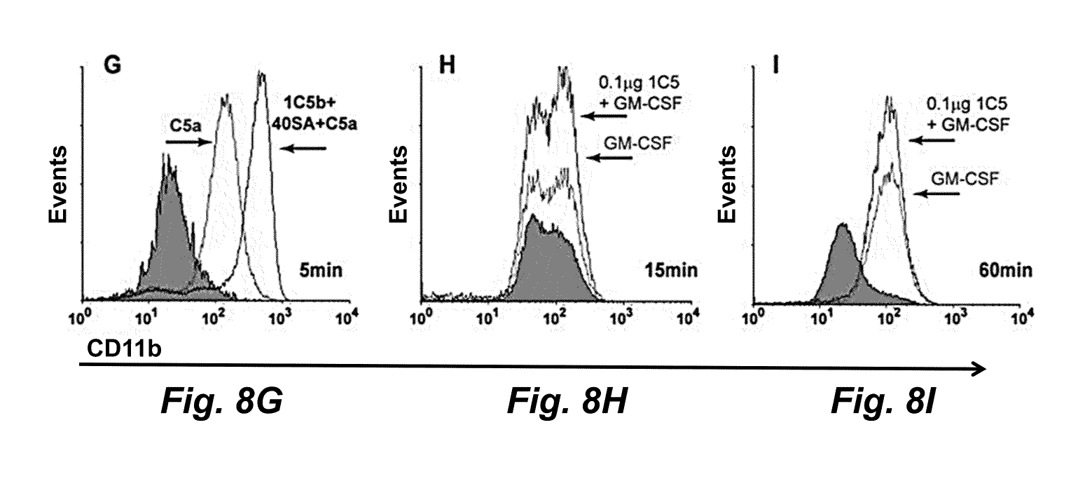

As seen in FIG. 8A, ligation of TLT2 alone has no effect on CD11b expression on purified neutrophils, regardless of the mAb concentration used, compared to the FMLP receptor agonist WKYMVm, which induced CD11b expression in a dose dependent manner (FIG. 8B). However, when neutrophils were pre-incubated with 100 ng/mL of 1H4 mAb and were subsequently stimulated with 1 nM WKYMVm, a concentration that does not significantly alter CD11b expression, a synergistic up-regulation of CD11b was observed (FIG. 8C). As was seen in the ROS assay, the potentiation of CD11b expression in response to FMLP following TLT2 ligation was further enhanced when neutrophils were pre-incubated in the presence of biotinylated 1C5 mAb plus streptavidin as a secondary cross-linking agent (FIGS. 8D-8F). Exposure to C5a also results in neutrophil degranulation, and like FMLP, activation of cells by C5a is the result of signal transduction mediated by the C5a receptor, a GPCR. Therefore experiments were performed to determine if TLT2 ligation would potentiate the cellular response to C5a. As seen in FIG. 8G, pre-incubation of neutrophils with anti-TLT2 mAb potentiates degranulation in response to C5a resulting in the up-regulation of CD11b. In contrast, ligation of TLT2 does not affect CD11b up-regulation when used in conjunction with GM-CSF (FIGS. 8H and 8I). Pre-treatment of neutrophils with isotype control mAb alone or in conjunction with any of the agonists tested did not alter CD11b expression.

### TLT2 Ligation Potentiates Neutrophil Degranulation and Up-Regulation of the Activation Marker CD11b in Response to GPCR-Mediated Signaling:

To determine if ligation of TLT2 potentiates the ability of TLR agonists to induce degranulation of neutrophils, experiments were performed in which neutrophils were pre-incubated in the presence of αTLT2 or isotype control mAb followed by addition of TLR agonists, including LPS, poly(I:C), MPLA, flagellin, imiquinod and CpG-ODN, which bind to TLR2, 3, 4, 5, 7/8 and 9, respectively. Neutrophil degranulation in response to these TLR agonists was not affected by pre-incubation of neutrophils with αTLT2 or isotype control mAb. A second assay was performed to monitor the effect that TLT2 ligation has on the ability of TLR agonists to prime neutrophil degranulation in response to low concentrations of GM-CSF. TLT2 ligation had no effect on the priming activity of any of the TLR agonists examined.

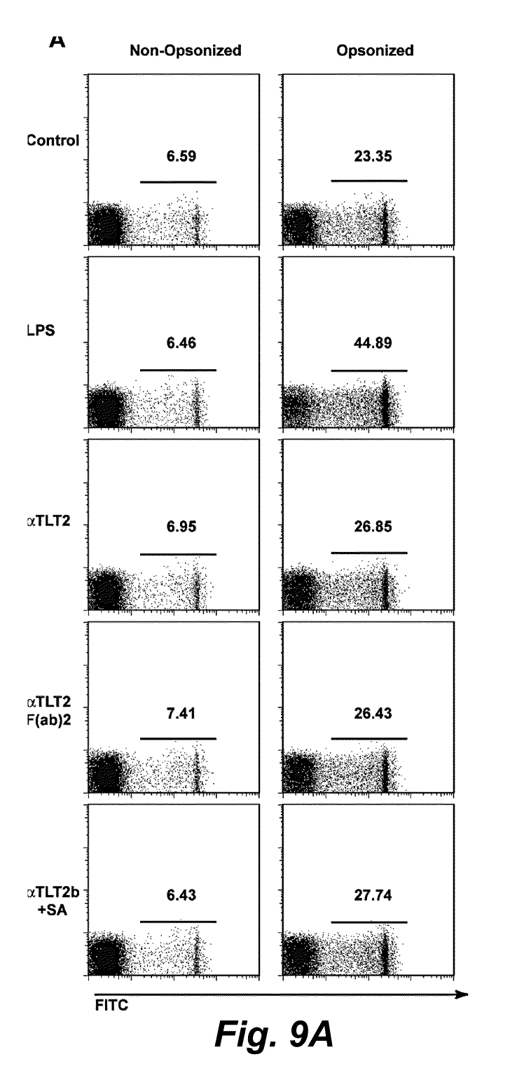

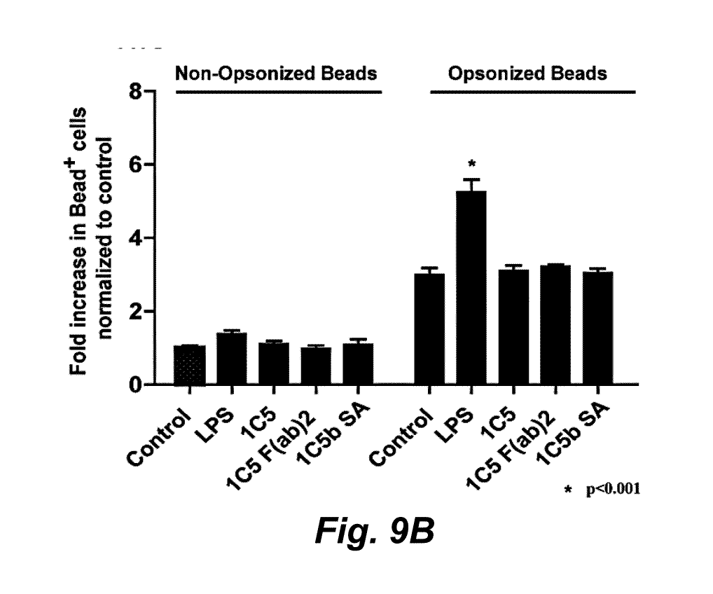

The fact that TLT2 ligation specifically potentiates the response of neutrophils to FMLP, a bacterial component, and is up-regulated on neutrophils at sites of inflammation suggests that it may play an important role in the innate immune response to bacterial infection. Phagocytosis of bacteria by neutrophils is mediated by multiple receptors including integrins, lectins, and scavenger receptors (Underhill et al., (2002) Annu. Rev. Immunol. 20: 825). However, the phagocytosis of bacteria by neutrophils is greatly enhanced in the presence of opsonizing antibodies, which promote engulfment of particles via Fc receptors (FcR). Thus experiments were performed to determine if the FcR-dependent phagocytic activity of neutrophils is enhanced by ligation of TLT2. To experimentally determine the effect of TLT2 ligation on the engulfment of opsonized particles, two-micron polystyrene beads were conjugated to FITC to facilitate visualization, and then incubated with purified neutrophils either alone or following opsonization with polyclonal αFITC antibody. After 15 min, trypan blue was added to quench extracellular FITC fluorescence and phagocytosis was assayed by flow cytometry. As seen in FIG. 9, ligation of TLT2 had no measurable effect on the percentage of neutrophils that phagocytosed beads via a FcR-dependent mechanism, as αTLT2 mAb pretreated samples were identical to non-pretreated controls, regardless of whether intact mAb, F(ab′)₂ fragments, or secondary cross-linking was used.

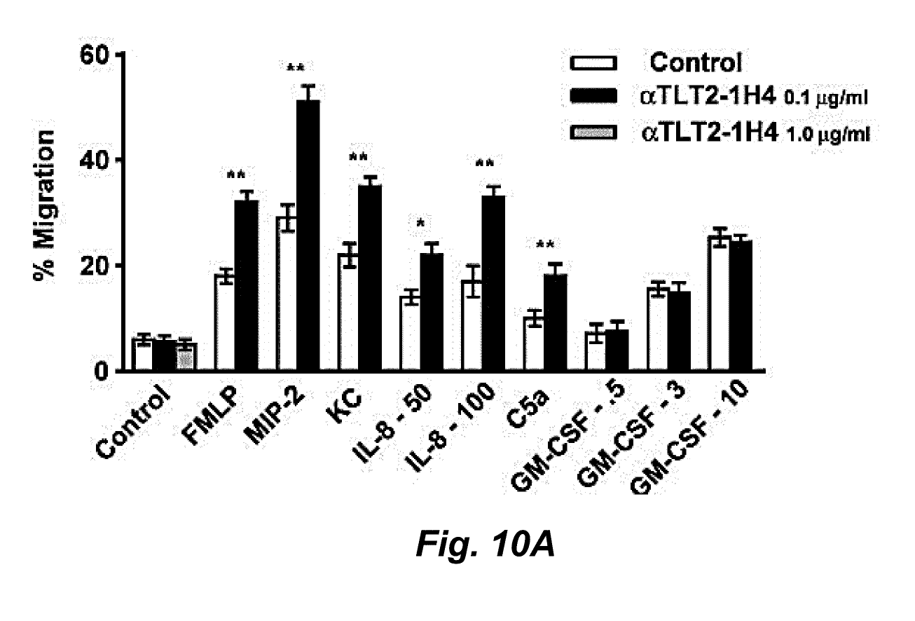

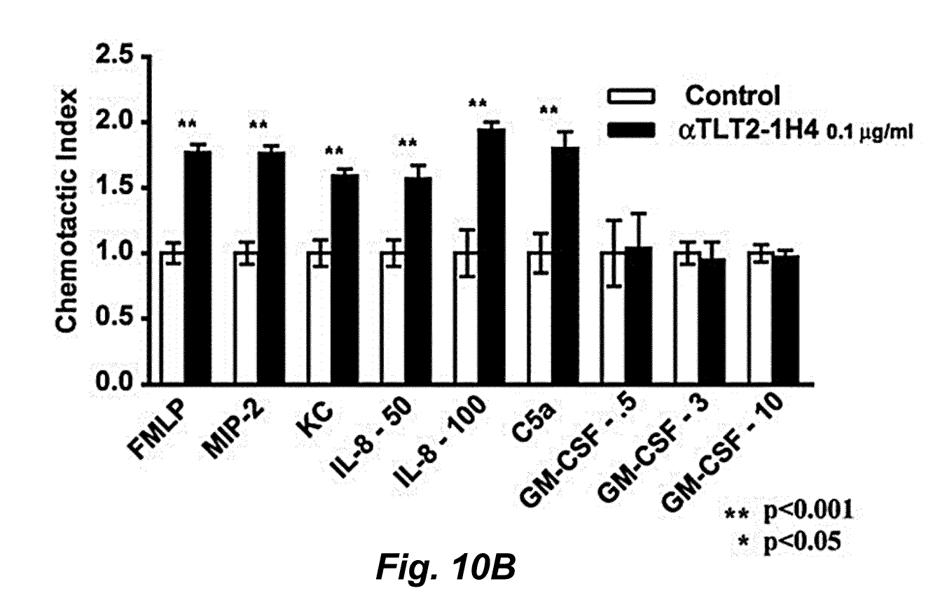

TLT2 ligation potentiates the functional response of neutrophils to FMLP and C5a, including cell migration, ROS production, and up-regulation of CD11b, but exhibits no effect on neutrophil functional responses to TLR agonists, GM-CSF or FcR-mediated activation. Given the important role that GPCRs play in regulating neutrophil migration in vivo, it was of interest to determine if TLT2 ligation potentiates the migration of these cells in response to other chemokines. Chemotaxis of purified neutrophils was assayed in response to a variety of chemoattractants including the murine chemokines KC and MIP-2, their human homologue IL-8, as well as the activated complement component C5a. In this assay, TLT2 ligation in the absence of chemoattractant had no measurable effect. However, in the presence of chemokine, αTLT2 mAb resulted in a 1.7- to 2.0-fold increase in the number of cells that migrate in response to these chemoattractants (FIGS. 10A and 10B). This effect is not observed in response to GM-CSF, which is the only member of this panel of chemoattractants that does not signal via a GPCR. Treatment with isotype control mAb had no effect on neutrophil chemotaxis nor did it potentiate migration in response to any of the chemoattractants tested (FIGS. 7A-7D). The percentages of cells migrating in these assays varied depending on the chemoattractant utilized, the magnitude of the effect associated with TLT2 ligation was constant. This demonstrates that TLT2 ligation potentiates the response of neutrophils to multiple GPCRs, as the effect of TLT2 ligation is similar whether neutrophils are responding to signals delivered via the FMLP receptor, CXCR2, or the C5a receptor.

### TLT2 Ligation Promotes Neutrophil Recruitment to Sites of Inflammation In Vivo:

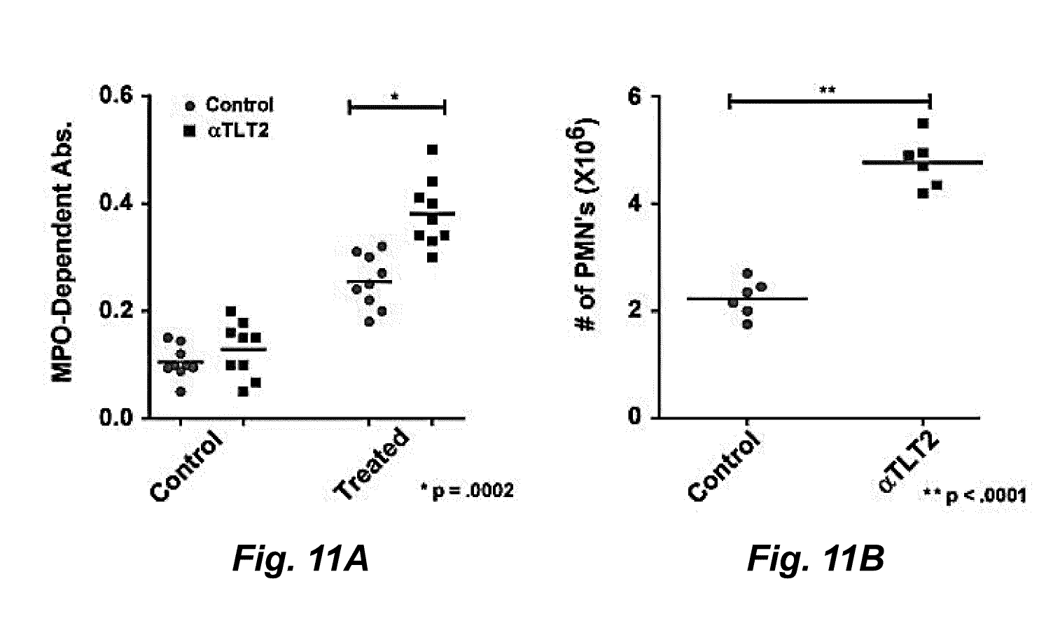

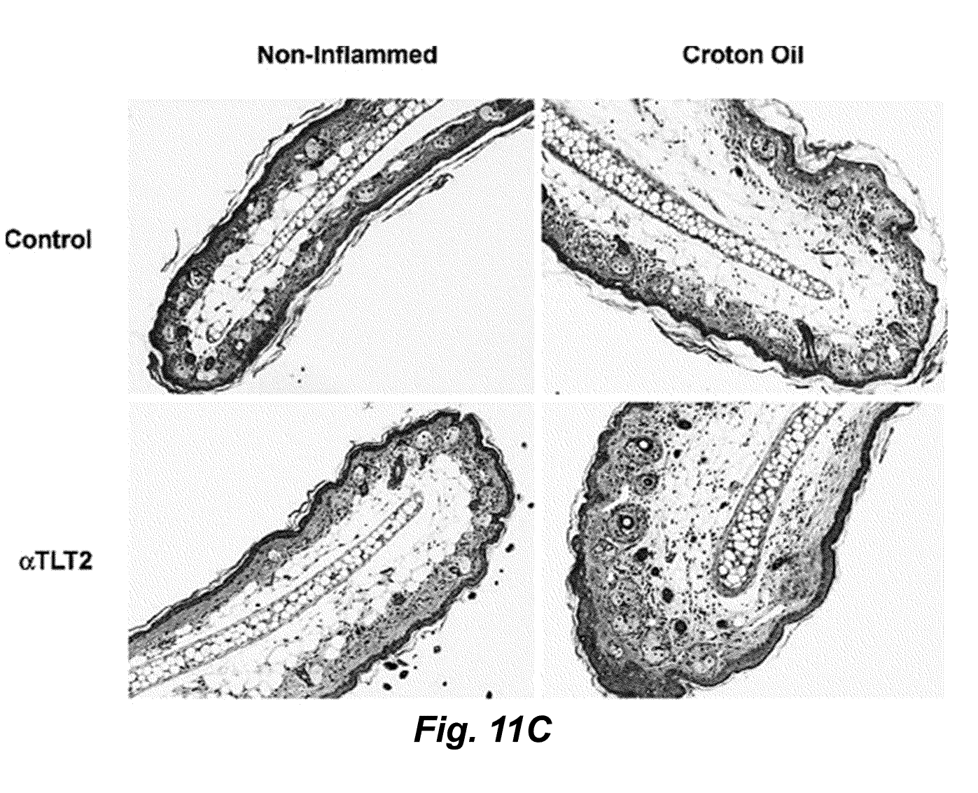

Because ligation of TLT2 potentiates the chemotactic response of neutrophils to agonists that bind GPCRs, and given the importance of these receptors in controlling the migration and recruitment of neutrophils, it was of interest to determine if TLT2 ligation potentiates neutrophil recruitment to sites of experimentally-induced inflammation in vivo. Croton oil (2% in acetone) was used to induce a nonspecific inflammatory response in the ear. Thirty minutes following i.v. administration of either 1H4 mAb in the experimental group, or saline in control mice, croton oil was applied to the pinna of one ear while acetone alone was applied to the other. After 4 h the mice were sacrificed and a 4 mm biopsy was removed from both ears. These biopsies were mechanically dissociated and the resulting lysate was analyzed for the presence of MPO (FIG. 11A) using the colorimetric substrate tetramethylbenzidine (TMB) (Menegazzi et al., (1992) J. Leukoc. Biol. 52: 619).

Assaying the MPO activity present in these samples against standard curves of horse radish peroxidase (HRP) facilitated the estimation of the number of neutrophils present in these samples. As seen in FIG. 11B, administration of 1H4 mAb resulted in a two-fold increase in the number of neutrophils present in the inflamed ears, whereas there was no significant difference in the number of neutrophils present in samples derived from non-inflamed ears. To further examine the effect that administration of αTLT2 mAb in vivo had on neutrophil recruitment and the inflammatory process in control and croton oil-treated ears, H&E staining of sections taken from the ear was performed to assess the cellular infiltrate as well as to examine the architecture of the ear.

As seen in FIG. 11C, administration of αTLT2 mAb increased the recruitment of neutrophils to croton oil-treated ears in comparison to mice that received a sham injection of saline. The cellular influx consisted mainly of neutrophils in both treated and control animals based on examination of the sections at high magnification. Administration of αTLT2 mAb had no effect on the recruitment of neutrophils to the control ear (FIG. 11C) and as demonstrated by an analysis of MPO activity (FIG. 11A). Finally, examination of the architecture of the ear did not reveal any significant differences at 3 h between the control and αTLT2-treated animals for either the control or croton oil-treated ear. These data support the conclusion that TLT2 engagement in vivo results in an enhanced chemotactic response to signals generated during inflammation thereby increasing the number of neutrophils accumulating in the inflamed tissue.

### Administration of αTLT2 mAb Results in Enhanced Accumulation of Neutrophils at Sites of Inflammation In Vivo:

Because TLT2 is broadly expressed by immune cells, including macrophages, neutrophils, and B lymphocytes, it is difficult to determine if enhanced recruitment of neutrophils following administration of αTLT2 mAb is the result of a direct effect of TLT2 ligation on neutrophils or due to an indirect effect resulting from TLT2 ligation on other cell types.

To determine if the observed enhancement of neutrophil accumulation at sites of inflammation is a direct effect of TLT2 ligation on responding neutrophils in vivo, a series of adoptive transfer experiments were performed in which neutrophils were isolated from the bone marrow of CD45.1 C57Bl/6 mice and labeled with either CFSE or CMPTX, or left unlabeled. These cells were then treated either with isotype control antibodies, F(ab′)₂ fragments of 1H4 αTLT2 mAb or intact 1H4. An equal mixture of treated cells was then adoptively transferred into CD45.2 C57Bl/6 recipient mice, which had been challenged intra-tracheally with 5 μg of LPS to induce inflammation in the lung 2 h prior to adoptive transfer. Four hours after adoptive transfer of treated neutrophils, the recipient mice were sacrificed, and cells present in the lung were isolated by lavage, stained with the appropriate antibodies, and analyzed by flow cytometry.

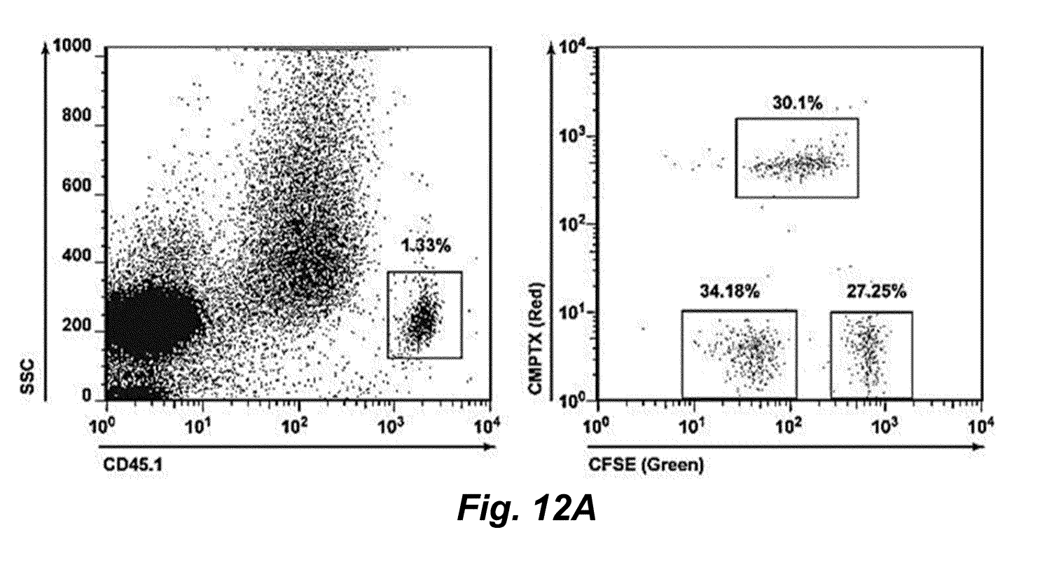

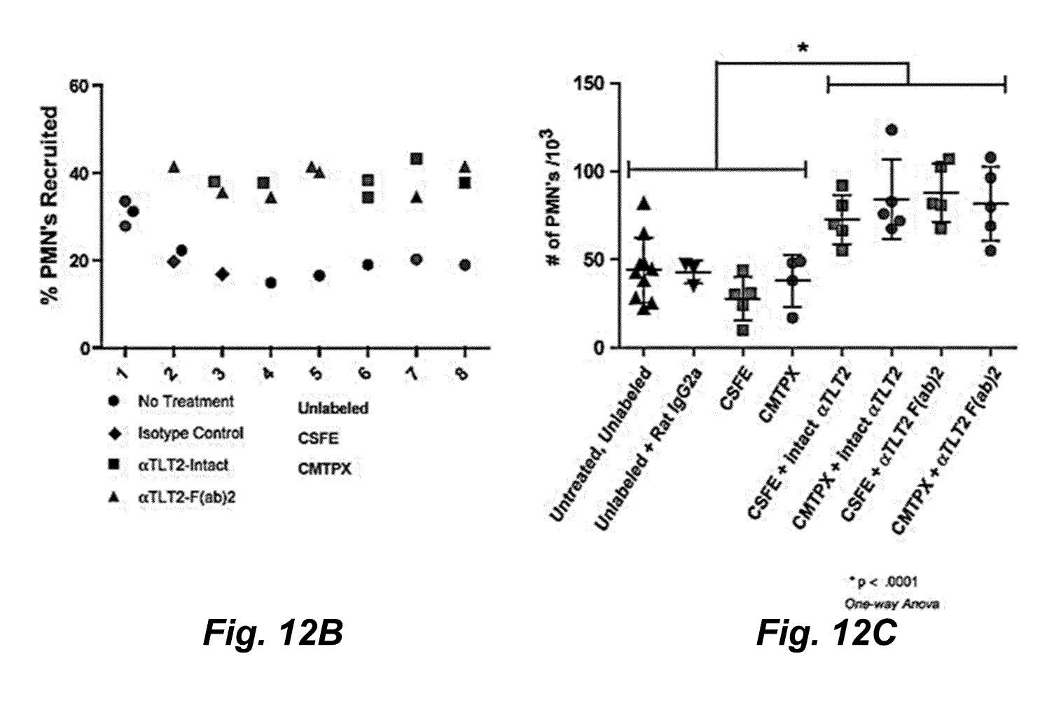

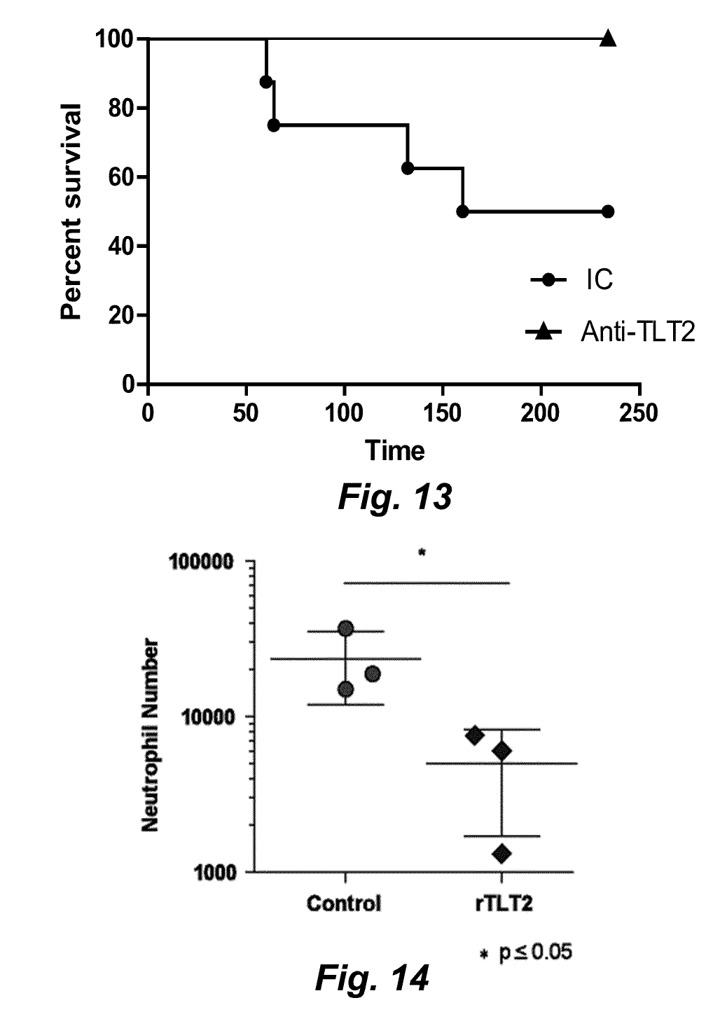

Adoptively transferred neutrophils were discriminated by the expression of CD45.1, and these cells were then analyzed based on the dye they were loaded with; green, red, or unlabeled, allowing for the identification of these populations present in the lungs of recipient mice (FIG. 12A). As shown in FIG. 12B, neither pre-treatment with isotype control antibody nor dye loading affected the recruitment of adoptively transferred neutrophils into the inflamed lung. Pre-treatment with αTLT2 mAb, however, increased the frequency of treated neutrophils recovered in comparison to untreated or isotype control cells, regardless of the labeling conditions (FIG. 12B). The difference in frequencies between controls and αTLT2 mAb treated neutrophils resulted in approximately a two-fold increase in the absolute number of αTLT2 treated cells isolated from the lungs of the recipient mice (FIG. 12C). The observed increase in the number of neutrophils that migrated occurred whether neutrophils were treated with intact 1H4 mAb or F(ab′)₂ fragments of this mAb. As these experiments utilized pooled neutrophils isolated from the same group of donor animals and represent a specific enhancement in the recruitment of αTLT2 mAb-treated neutrophils compared to control cells within the same recipient animal, these data strongly support the conclusion that ligation of TLT2 potentiates the chemotactic response of neutrophils to chemokines in vivo via a direct effect on the neutrophil itself.

### TLT2 Ligation Potentiates Chemokine and Growth Factor Production in Response to Inflammation:

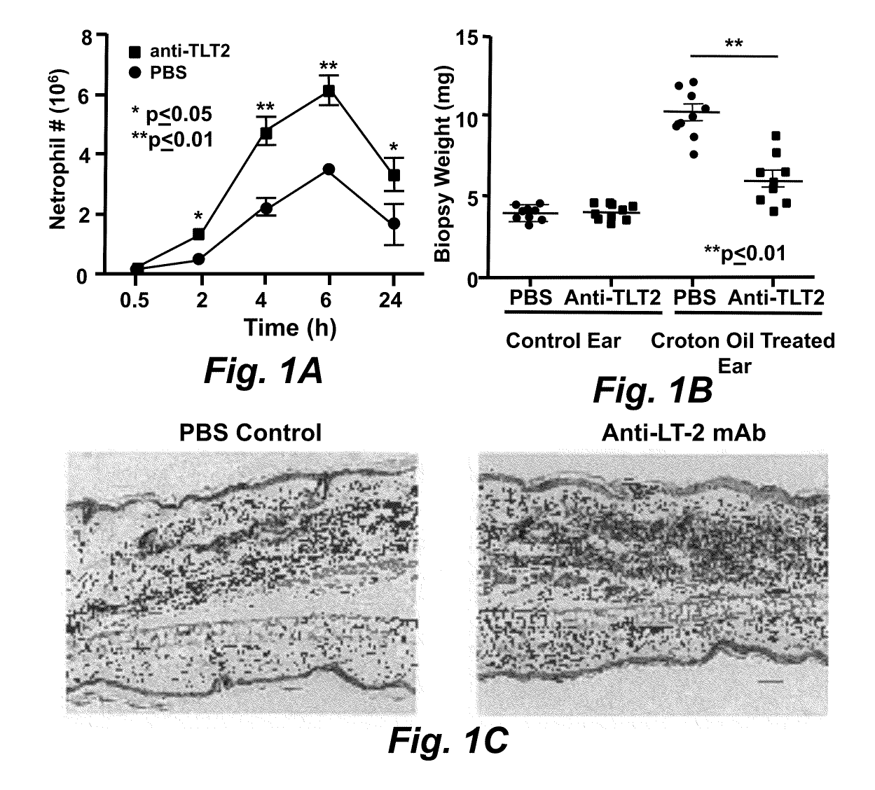

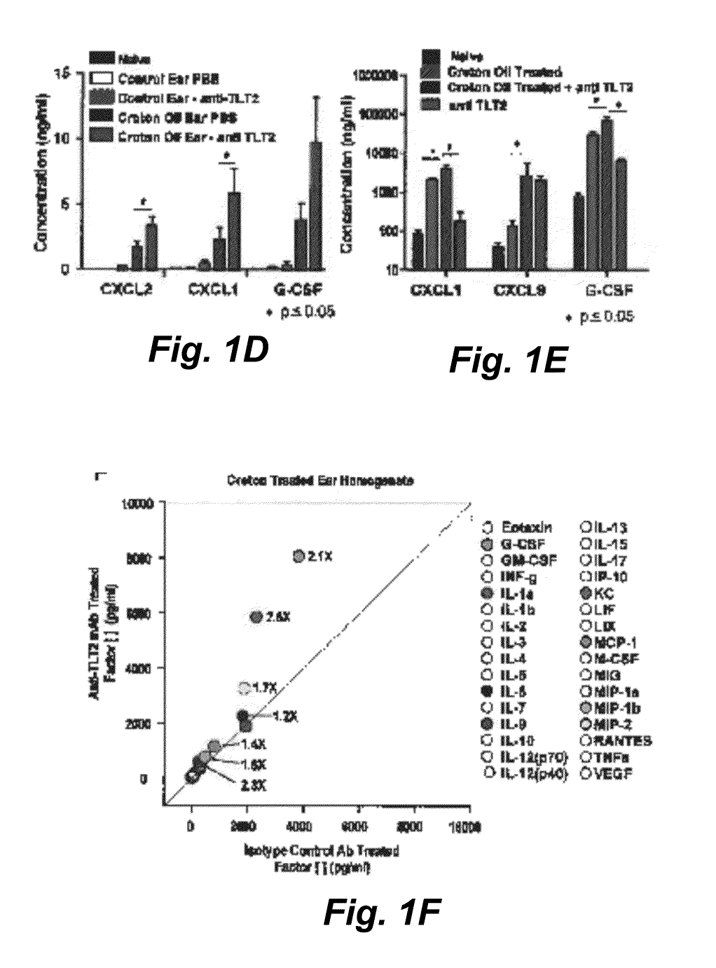

Previous studies have demonstrated that ligation of TLT2 on murine neutrophils results in an enhanced response of these cells to signals delivered via GPCRs, including the migratory response to the chemoattractants CXCLI and CXCL2. As depicted in FIG. 1A, the administration of anti-TLT2 mAb 30 min prior to the application of croton oil to the ears of mice resulted in an increased number of neutrophils participating in the inflammatory response as estimated by MPO quantification in ear biopsies (FIG. 1A). In addition to containing a higher number of neutrophils, biopsies from the ears of mice receiving anti-TLT2 mAb prior to croton oil treatment exhibited a decrease in mass compared to control animals (FIG. 1B). Moreover, the inflamed ears from anti-TLT2 mAb treated animals exhibited noticeable bruising that was absent in PBS treated animals.

Histological sections of ears prepared 12 h after the application of croton oil revealed a significant increase in cellular infiltrates consisting predominantly of neutrophils in anti-TLT2 mAb-treated animals (FIG. 1C). It was also noted that significant numbers of red blood cells were present in the tissue of anti-TLT2 mAb-treated animals consistent with the observed bruising (FIG. 1C). The increased influx of neutrophils, increased bruising, red blood cell deposition, and the decreased ear mass observed in anti-TLT2 mAb treated mice, suggested that TLT2 ligation significantly enhanced the overall inflammatory response. Because TLT2 ligation has been shown to potentiate neutrophil activation in response to GPCR-mediated signals, it is possible that the increased tissue damage, including disruption of the microvasculature, was solely due to TLT2-dependent potentiation of neutrophil recruitment and activation. Alternatively, because TLT2 is expressed on other hematopoietic cells, including macrophages and B lymphocytes, it is possible that administration of anti-TLT2 mAb contributed to the inflammatory process via neutrophil extrinsic mechanisms through its action on other cell types.

Experiments were performed in which control or croton oil-treated ear homogenates from mice that had received anti-TLT2 mAb or PBS were assayed at 4 h to measure chemokines, growth factors and cytokines by Bio-Plex. It was determined that i.v. administration of anti-TLT2 mAb significantly enhanced the level of the chemokines CXCLI and CXCL2, and the growth factor G-CSF in croton oil-treated ears, compared to PBS control mice (FIG. 1D). In contrast little or no chemokine or growth factor could be detected in the control ears, regardless of whether the mice were injected with anti-TLT2 mAb.

Analysis of the blood 4 h after administration of croton oil revealed that the levels of CXCLI, CXCL9 (MIG) and G-CSF were significantly elevated in mice that received anti-TLT2 mAb (FIG. 1E). The effect that TLT2 ligation has on the production of soluble factors in vivo is restricted in nature as no other significant differences, with the exception of IL-6, were observed in croton oil-treated ear homogenates from mice that received anti-TLT2 mAb versus PBS (FIG. 1F). These data support the conclusion that TLT2 ligation in vivo enhanced the overall inflammatory response by potentiating the production of growth factors and chemokines that mediate neutrophil production, migration and activation.

Specific factors were detected in the blood of mice receiving anti-TLT2 mAb in the absence of an acute inflammatory stimulus. Significant levels of CXCL9 and G-CSF production were detected in the blood following administration of anti-TLT2 mAb, indicating that ligation of TLT2 alone was sufficient to induce the production of chemokines and growth factors. In the case of G-CSF, treatment with anti-TLT2 mAb and croton oil appear to synergistically increase the concentration of G-CFS present in the blood, resulting in a G-CSF concentration that was 2-fold higher than that measured in mice receiving only croton oil, and 10-fold higher than in mice that received anti-TLT2 m.Ab alone. These data demonstrate that ligation of TLT2 potentiates the inflammatory response through multiple mechanisms that include its direct effect on neutrophils thereby enhancing their response to signals delivered via GPCRs, as well as the potentiation of growth factor and chemokine production in concert with inflammatory conditions.

### TLT2 Ligation Promotes Neutrophil Recruitment in the Absence of Inflammatory Stimuli:

The observation that i.v. administration of anti-TLT2 mAb results in an elevation of GCSF and CXCL9 in the blood, suggested that localized TLT2 engagement may promote the generation of chemokines and growth factors that affect neutrophil biology. Mice were injected either i.p. or i.t. with anti-TLT2 mAb or IC Ab. Peritoneal or bronchial lavages were performed and infiltrating cells were quantified by flow cytometry between 2-24 h after administration of the antibody.

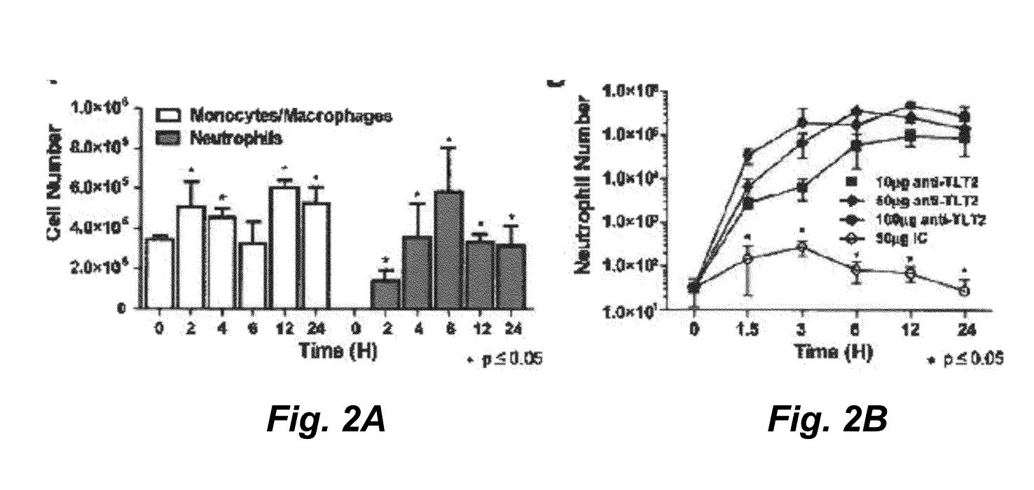

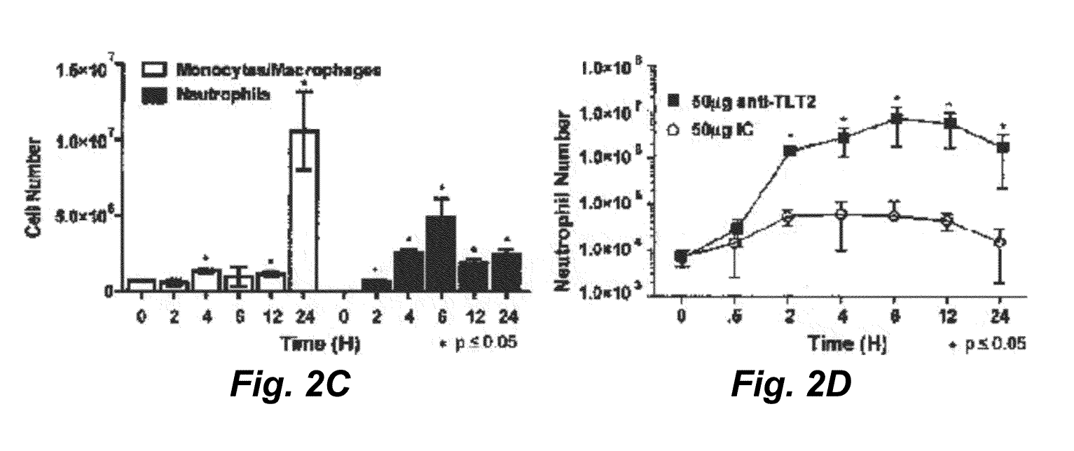

As seen in FIG. 2A, i.t. injection of anti-TLT2 mAb induced a modest, but statistically significant, increase in the number of alveolar macrophages present in the lung between 2-24 h. In contrast, TLT2 ligation induced the rapid recruitment of significant numbers of neutrophils within 2 h, reaching a maximum by 6 h. This effect is specific to anti-TLT2 mAb and is dose dependent, as intratracheal injection of IC Ab had little or no effect on neutrophil recruitment (FIG. 2B). Similarly, when anti-TLT2 mAb was injected i.p., it was observed to induce the rapid influx of neutrophils within 2-6 h, which was not observed in response to IC Ab (FIGS. 2C and 2D). As was observed in the lung, anti-TLT2 mAb did not appear to dramatically affect macrophage recruitment at early time points. However, there was a significant influx of macrophages at 24 h, suggesting that they were recruited in response to secondary processes triggered by TLT2 ligation. These data support the conclusion that administration of anti-TLT2 mAb drives the production of extrinsic factors that promote neutrophil activation and migration, possibly in conjunction with the neutrophil intrinsic action of TLT2, as previously shown (Halpert et al., (2011) J. Immunol. 187: 2346-2355).

### TLT2 Ligation Induces Production of Chemokines and Growth Factors in the Absence of Other Stimuli In Vivo:

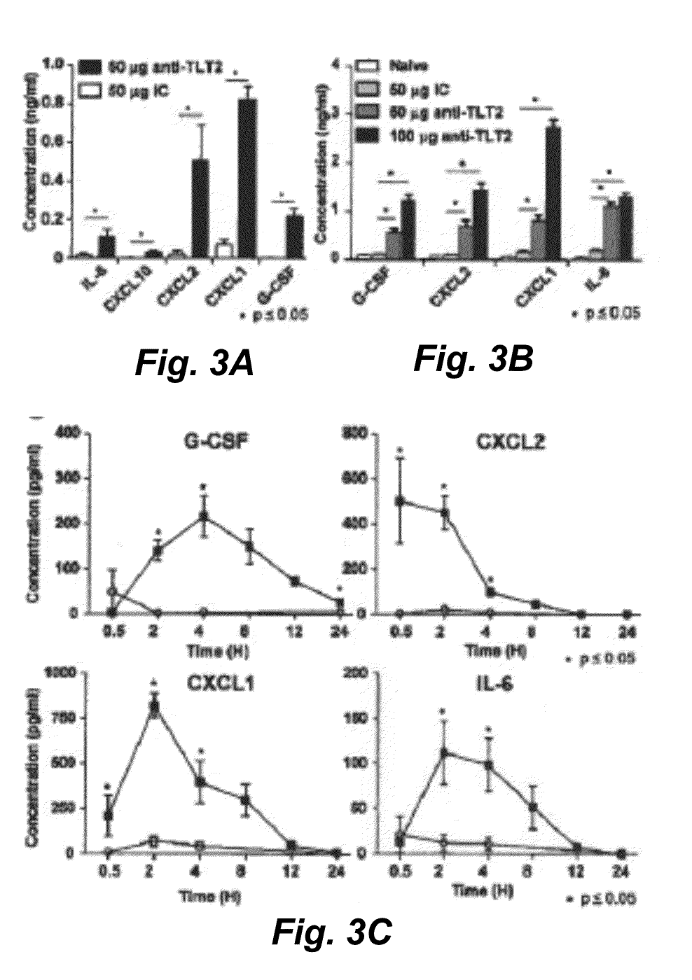

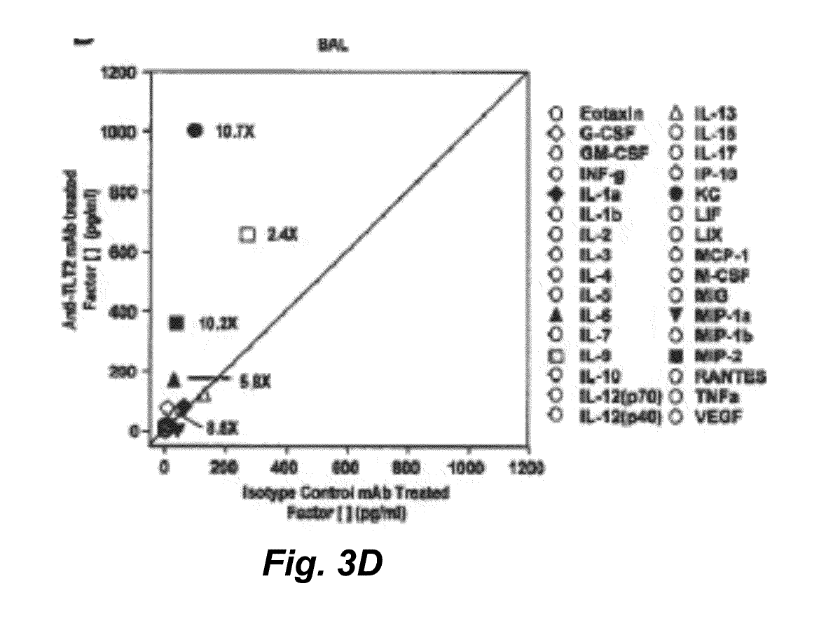

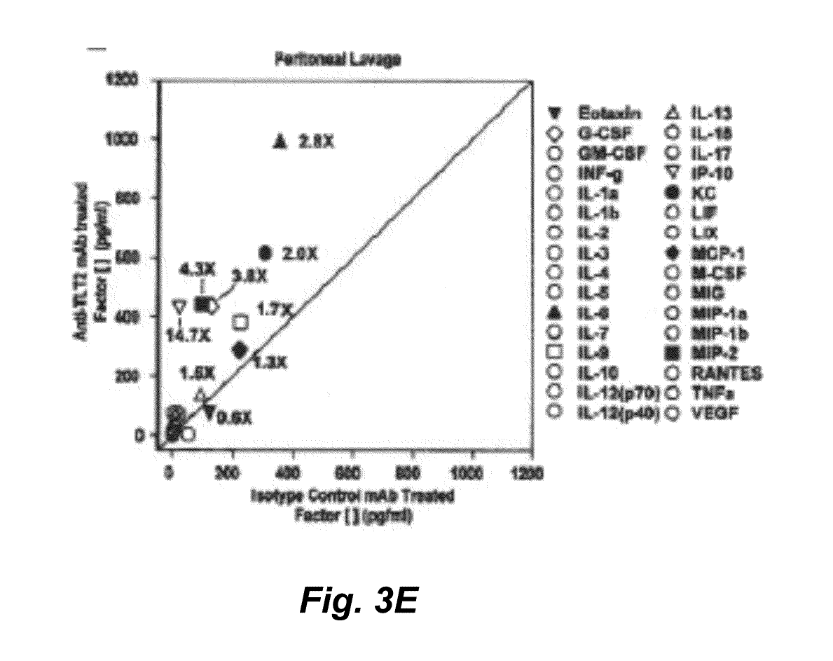

The production of growth factors, cytokines and chemokines induced by the ligation of TLT2 was monitored using the Bio-Plex assay. Of 32 analytes examined, anti-TLT2 mAb administration in the lung induced the production of a relatively restricted set of soluble factors including the growth factor G-CSF as well the chemokines CXCLI and CXCL2 (FIG. 3A).

The production of soluble factors in response to anti-TLT2 mAb exhibited a dose dependent response (FIG. 3B) and occurred rapidly within 30 min to 2 h of mAb administration (FIG. 3C). In general, the levels of most factors remained elevated for 2-8 h above the levels observed in mice that received IC Ab (FIG. 3C). The kinetics of the response to anti-TLT2 mAb supports the hypothesis that ligation of TLT2 mediates both neutrophil extrinsic as well as intrinsic effects because growth factor and chemokine production precedes the maximal recruitment of neutrophils to the lung.

Analysis of soluble factor production in the peritoneum after i.p. administration of anti-TLT2 mAb revealed that G-CSF, CXCLI and CXCL2 were also elevated compared to animals that received IC Ab (FIG. 3E). Similarly, the production of the cytokine IL-6 was elevated in both lung and peritoneum (FIGS. 3D and 3E), whereas other cytokines exhibited little change in anti-TLT2 mAb-treated mice compared to control animals (FIGS. 3D and 3E). In contrast to the factors that were produced at significant levels in both lung and peritoneum, CXCL10 (IL-10) was produced predominantly in the peritoneal cavity in response to TLT2 ligation (FIG. 3E).

TLT2 Ligation on Resident Macrophages Induces the Production of Factors that Regulate Neutrophil Production and Chemotaxis:

Because administration of anti-TLT2 mAb in vivo was observed to induce the production of growth factors and chemokines that affect neutrophil production and migration, it was of interest to determine what cell types are responsible for producing these factors. A logical cell population to examine in this regard is the resident macrophage population as these cells express significant levels of TLT2 on their surface. Therefore, purified resident peritoneal macrophages were stimulated with soluble F(ab′)₂ fragments of anti-TLT2 mAb or IC Ab for various periods of time and the supernatants were harvested for Bio-Plex analysis.

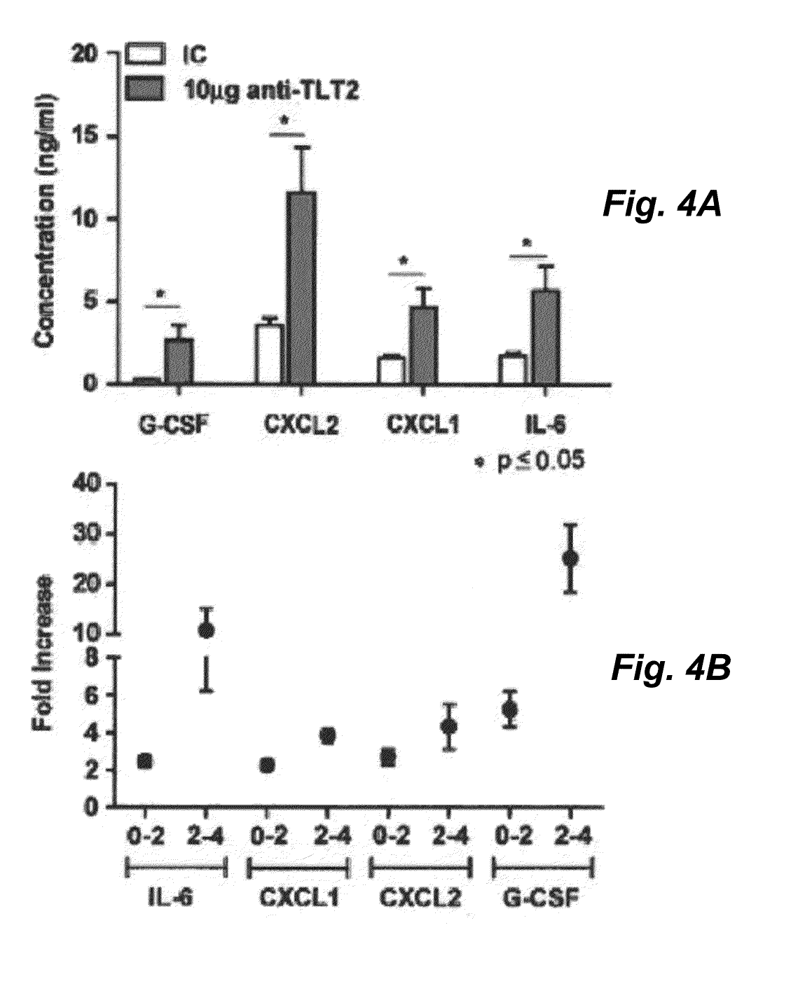

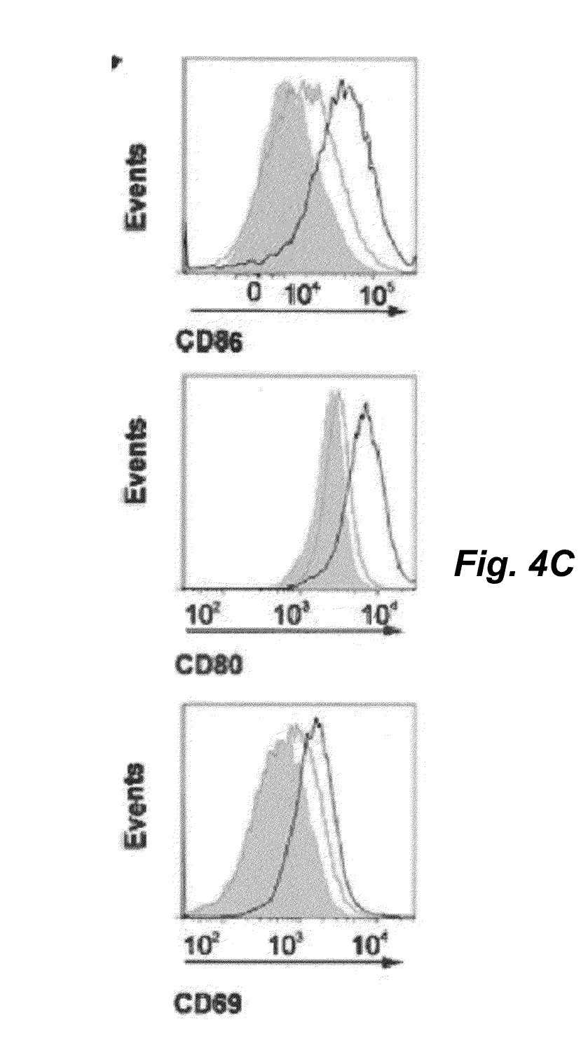

It was found that TLT2 ligation on resident peritoneal macrophages induced the production of GCSF, CXCLI, CXCL2, and IL-6, which are all produced in vivo in response to administration of anti-TLT2 mAb (FIG. 4A). As was observed in vivo, ligation of TLT2 on resident macrophages in vitro drives the rapid production of all factors within the first 2 h, leading to significant accumulation of factors by 4 h compared to IC Ab-treated cells (FIG. 4B). This finding is striking as previous studies examining the effect of TLT2 on neutrophils suggested that TLT2 ligation in the absence of GPCR agonists had no effect on the activation or migration of these cells (Halpert et al., (2011) J. Immunol. 187: 2346-2355). Indeed, TLT2 ligation on neutrophils in the absence of other stimuli does not induce the production of soluble factors, in contrast to what is observed for resident macrophages. Thus, it appears that TLT2 ligation on macrophages drives a qualitatively different signal that leads to the production of chemokines and growth factors that act on neutrophils. Importantly, based on analysis of activation marker expression including CD69, CD80, and CD86. it does not appear that TLT2 ligation on macrophages leads to generalized activation of these cells (FIG. 4C).

TLT2 has been shown to be expressed on neutrophils, macrophages and B cells, which is a unique characteristic compared to other TREM locus receptors, suggesting that it may play a role in regulating aspects of both the innate and adaptive immune response (King et al., (2006) J. Immunol. 176: 6012-6021). It has been shown that ligation of TLT2 neutrophils potentiates the cellular response to a range of agonists that signal via GPCRs. Ligation of TLT2 enhances ROS production, degranulation and migration of neutrophils in response to the formylated peptide fMLF. With respect to its ability to potentiate neutrophil functional responses, studies demonstrated that TLT2 synergizes with FPR-1, C5aR and CXCR2, all of which are coupled to G_{αi} subclass proteins, to enhance neutrophil responses. In contrast, TLT2 was not observed to potentiate neutrophil function in response to growth factor receptor (CSF3), FcR, or TLR dependent signals. Thus, TLT2 appears to selectively regulate the neutrophil response to a range of GPCR agonists. TLT2 ligation alone was not observed to induce ROS production, degranulation or migration in the absence of GPCR agonists, indicating that its main function is to potentiate the cellular response to GPCR-mediated signals.

Because TLT2 expression on neutrophils and macrophages is up-regulated in vivo in response to inflammatory conditions (King et al., (2006) J. Immunol. 176: 6012-6021) and it potentiates neutrophil migration as well as activation, it was considered that ligation of TLT2 vivo may potentiate the acute inflammatory response. It was shown, however, that TLT2 ligation potentiated neutrophil recruitment to local sites of inflammation, presumably due to a direct effect on the neutrophil response to chemotactic factors.

Increased neutrophil recruitment following TLT2 ligation was associated with loss of tissue mass and increased bruising resulting from disruption of the microvasculature. Because TLT2 is expressed on macrophages and B cells it was considered that its role in enhancing acute inflammation could be due to its direct effect on neutrophils, as well as indirect effects in which its ligation modulates the function of other cell types. In this regard, it was found that TLT2 ligation in vivo potentiates the production of soluble factors, including G-CSF and the chemokines CXCL1 and CXCL2 that act on neutrophils. The production of these factors is likely to contribute to the overall inflammatory response through direct effects on neutrophils, as well as through their effects on the vascular endothelium. The chemokines CXCL1 and CXCL 2 play an important role in neutrophil recruitment (Kobayashi, Y. (2008) Front. Biosci. 13: 2400-2407; Pelus et al., (2002) Crit. Rev. Oncol. Hematol. 43: 257-275), but also have the ability activate neutrophils, as does G-CSF, leading to increased degranulation. The CXC family of chemokines, including CXCL1, CXCL2 and CXCL8 also act directly on endothelial cells via CXCR2 to promote endothelial cell retraction, which results in decreased barrier function and increased vascular permeability (DiStasi & Ley (2009) Trends Immunol. 30: 547-556). Thus, TLT2-dependent potentiation of inflammation is mediated through its direct effect on neutrophils as well as effects on other cell types leading to the elaboration of soluble factors. The production of CXCL1 and CXCL 2 in turn potentiate neutrophil migration and activation, as well as modulation of endothelial cell function, further increasing the magnitude of the inflammatory response (DiStasi & Ley (2009) Trends Immunol. 30: 547-556).

During the course of the croton oil experiments as disclosed herein, it was found that i.v. administration of anti-TLT2 mAb alone potentiated the production of selected soluble factors (e.g. CXCL1 and GCSF) in the blood, suggesting that TLT2 ligation in vivo is sufficient to drive many of the events observed in the context of acute inflammation. This was further supported by experiments demonstrating that administration of anti-TLT2 mAb in the lung or peritoneum induced rapid and sustained neutrophil migration in the absence of other stimuli. Because it has been shown that ligation of TLT2 on neutrophils in the absence of GPCR-mediated signaling has no effect on neutrophil activation or migration, it was evident that administration of anti-TLT2 mAb in vivo promotes neutrophil recruitment via the activation of neutrophil extrinsic processes. Indeed, it was determined that injection of anti-TLT2 mAb induces the production of the growth factor G-CSF, which plays a critical role in promoting neutrophil production as well as certain aspects of neutrophil activation, including degranulation (Furze & Rankin (2008) Immunology 125: 281-288; Panopoulos & Watowich (2008) Cytokine 42: 277-288). Additionally, the chemokines CXCL1 and CXCL2, which play a predominant role in the recruitment of neutrophils (Kobayashi, Y. (2008) Front. Biosci. 13: 2400-2407; Pelus et al., (2002) Crit. Rev. Oncol. Hematol. 43: 257-275) were significantly elevated in anti-TLT2 mAb-treated mice.

Although neutrophils can elaborate CXCL1 and CXCL2 (McColl at al., (2006) FASEB J. 20: 187-189; Scapini et al., (2000) Immunol. Rev. 177: 195-203), treatment of neutrophils ex vivo with anti-TLT2 mAb failed to elicit the production of soluble factors based on Bio-Plex analysis. This does not categorically rule out the possibility that ligation of TLT2 on neutrophils in vivo can elicit soluble factor production, but it does suggest that this is not the case. In contrast, it was shown that resident macrophages are at least in part responsible for the production of soluble factors that regulate neutrophil production, activation and migration in response to administration of anti-TLT2 mAb. Ligation of TLT2 on resident macrophages ex vivo in the absence of other stimuli resulted in the production of several factors observed in vivo in response to administration of anti-TLT2 mAb, including G-CSF, CXCL1, CXCL2, and IL-6. The production of soluble factors by macrophages was not associated with the general activation of these cells based on analysis of activation marker expression.

The Trem locus encodes several receptors expressed on myeloid lineage cells that function to modulate the response of cells to ligands for PRRs. In the case of TREM-1, ligation potentiates the systemic inflammatory response following stimulation of cells via TLRs and in some instances can exert a direct effect on macrophages leading to the production of inflammatory cytokines (Netea et al., (2006) J. Leukoc. Biol. 80: 1454; Bouchon et al., (2001) Nature 410:1103; Sharif & Knapp S. (2008) Immunobiology 213: 701; Colonna (2003) J. Infect. Dis. 187: S397). In contrast, TREM-2, which is expressed on a range of cell types, including macrophages has been shown to exert an inhibitory effect on TLR-mediated signaling resulting in attenuation of activation and cytokine production. TLT2 is expressed on cells of the myeloid lineage, including neutrophils and macrophages, and is up-regulated in response to inflammatory conditions in vivo (King et al., (2006) J. Immunol. 176: 6012). Based on these observations, it was of interest to determine if TLT2, like other members of the TREM family, modulates the functional response of myeloid lineage cells to signals derived from PRRs.

Accordingly, the present disclosure now provides evidence that TLT2 ligation potentiates ROS production, degranulation and chemotaxis in response to numerous GPCR agonists, including FMLP, C5a, MIP-2, KC and IL-8. TLT2 ligation was not observed to potentiate the cellular response to GM-CSF receptor or FcR-mediated signaling, which involve reversible protein tyrosine phosphorylation. Moreover, TLT2 ligation did not potentiate neutrophil degranulation in response to TLR agonists. Thus, TLT2 appears to mediate signaling processes that selectively potentiate the response to GPCRs. Importantly, ligation of TLT2 was not observed to decrease the threshold of sensitivity to GPCR agonists, supporting the conclusion that may act by amplifying or prolonging the GPCR signal, which in turn potentiates the functional response of the cell. Alternatively, because studies have yet to be performed demonstrating that TLT2 ligation affects the qualitative or quantitative nature of the biochemical signals associated with GPCRs, it is formally possible that TLT2 initiates a parallel signaling pathway that amplifies the functional response to GPCRs without directly intersecting those pathways. Regardless, the role of TLT2 as a receptor that potentiates the response of neutrophils to GPCR-mediated signals is comparable to the functions of TREM-1 and TREM-2 which modulate TLR-mediated signaling.

TLT2 does not possess charged transmembrane residues like TREM-1 and TREM-2 that would promote the interaction with transmembrane signaling effectors such as DAP12. Unlike TLT1, TLT2 does not contain an ITAM or ITIM in its cytoplasmic domain, which have the ability to recruit SH2 domain-containing effector proteins to mediate signal transduction (Ford et al., (2009) Curr Opin Immunol. 21: 38; Barrow (2004) J. Immunol. 172: 5838). Indeed, the cytoplasmic tail of human and mouse TLT2 are not highly conserved and there are no apparent signaling motifs that would, a priori, be predicted to be important for potentiating GPCR-mediated signaling. The TLT2 cytoplasmic domains from human and mouse are proline-rich and it is possible that this could confer the ability to interact with WW or SH3 domain-containing proteins. Alternatively, the proline residues may play a role in providing tertiary structure to the cytoplasmic domain that is important for mediating interactions with key effector proteins. Ligation of TLT2 does not potentiate the cellular response to signals delivered via the GM-CSF receptor, which is a receptor protein tyrosine kinase, or Fc receptors, which also engage protein tyrosine kinases. Moreover, TLT2 ligation does not appear to modulate the response to TLR signaling, as is the case for TREM-1 and TREM-2. Therefore, it appears that TLT2 selectively modulates the cellular response to GPCR-mediated signaling.

Because TLT2 is expressed on both neutrophils and macrophages, which are critical effectors of the innate immune response against bacteria, and its expression is increased on these cells in response to inflammation, the ligand for TLT2 may be derived from infectious organisms, or be generated as a byproduct of the innate immune response against pathogens (e.g. an acute phase protein). This is supported by the fact that TLT2 is most highly expressed on marginal zone and B1 B cell subpopulations, which are involved in mounting the natural/innate humoral immune response against bacterial pathogens.

Accordingly, it has been shown that TLT2 plays an important role in potentiating the neutrophil response to GPCR agonists leading to enhanced ROS production, degranulation and chemotaxis. Importantly, TLT2 does not appear to potentiate the cellular response to other types of receptors. Because TLT2 is up-regulated on myeloid lineage cells in response to inflammatory conditions in vivo, and plays a role in potentiating the cellular response against agonists derived from pathogens or generated during the innate immune response, it is clear that this member of the TREM locus is important in potentiating the anti-bacterial innate immune response via a novel mechanism that involves the potentiation of the response to GPCR signaling.

One aspect of these data of the disclosure therefore relates to the fact that administration of anti-TLT2 mAb in viva drives soluble factor production and potent neutrophil recruitment to the lung or peritoneal cavity in the absence of other stimuli. Because TLT2 is constitutively expressed on neutrophils, macrophages and B cells (King et al., (2006) J. Immunol. 176: 6012-6021), this argues that the ligand for TLT2 is regulated in terms of its expression and/or physical accessibility. If this were not the case, then it would be expected that constitutive activation of 1172-dependent processes would lead to increased neutrophil production, altered migration into tissues and ultimately activation of pro-inflammatory processes as is observed following administration of anti-TLT2 mAb. Because this does not occur in the absence of an inflammatory stimulus, it supports the conclusion that expression of the TLT2 ligand may be regulated by inflammation and/or infection. It has already been shown that TLT2 itself is up-regulated on neutrophils and macrophages in response to inflammatory stimuli in viva (King et al., (2006) J. Immunol. 176: 6012-6021), therefore complementary up-regulation of ligand expression may be important for controlling the function of TLT2.

Accordingly, the results of the disclosure support the conclusion that TLT2 plays a critical role in driving neutrophil activation and migration in viva via distinct, yet interrelated mechanisms that include the production of growth factors (G-CSF) and chemokines (CXCL1 and CXCL2), as well as potentiation of the neutrophil response to those factors. These findings represent a novel mechanism in which activation of the TLT2-dependent, positive feed-forward loop plays an important role in potentiating the acute inflammatory response.

One aspect of the disclosure, therefore, encompasses embodiments of a method for enhancing an innate immune response of an animal or human subject, the method comprising the steps of delivering to a cell or population of cells an effective amount of an agent that specifically interacts with a TREM-like transcript 2 (TLT2) transmembrane receptor of the cell or population of cells and potentiates neutrophil and/or macrophage activation and/or migration.

In embodiments of this aspect of the disclosure, the cell or population of cells can be a neutrophil or population of neutrophils, wherein said cell or population of cells are isolated from an animal or human subject, a cultured neutrophil or population of neutrophils; or a neutrophil or population of neutrophils in an animal or human subject.

In embodiments of this aspect of the disclosure, the cell or population of cells can be a macrophage or population of macrophages, wherein said cell or population of cells are isolated from an animal or human subject, a cultured macrophage or population of macrophages; or a macrophage or population of macrophages in an animal or human subject.

In embodiments of this aspect of the disclosure, the agent can be an antibody, or a fragment thereof, capable of specifically binding to an extracellular domain of TLT2 of a neutrophil or a macrophage.

In embodiments of this aspect of the disclosure, the antibody can be a monoclonal antibody or a fragment thereof.

In embodiments of this aspect of the disclosure, the agent delivered to the cell or population of cells can initiate ligation of the TLT2 thereon.

In embodiments of this aspect of the disclosure, the agent delivered to the cell or population of cells can induce the release of at least one cytokine by the cell or population of cells, wherein the at least one cytokine can be characterized as capable of interacting with at least one G-protein coupled receptor of a neutrophil, thereby inducing neutrophil activation and/or migration.

In embodiments of this aspect of the disclosure, the agent delivered to the cell or population of cells can be mixed with a pharmaceutically acceptable carrier.

Another aspect of the disclosure encompasses embodiments of a method of potentiating a neutrophil response to a G-protein coupled receptor signaling comprising delivering to a neutrophil an effective amount of an agent specifically interacting with TREM-like transcript 2 (TLT2) transmembrane receptor of the neutrophil.

In embodiments of this aspect of the disclosure, the neutrophil can be an isolated neutrophil, a cultured neutrophil, or is a neutrophil of an animal or human subject.

Yet another aspect of the disclosure encompasses embodiments of a method of modulating an inflammatory response of an animal or human comprising delivering to the animal or human subject a composition comprising an effective amount of an agent specifically interacting with a ligand of a TREM-like transcript 2 (TLT2) transmembrane receptor of a neutrophil or a macrophage.

In embodiments of this aspect of the disclosure, the agent can be a recombinant polypeptide comprising a domain specifically binding to a TLT2-binding ligand.

In embodiments of this aspect of the disclosure, the domain specifically binding to a TLT2 agonist can be an extracellular region of TLT2, or a fragment thereof.

In embodiments of this aspect of the disclosure, the agent delivered to the animal or human subject can be mixed with a pharmaceutically acceptable carrier.

In embodiments of this aspect of the disclosure, the method can further comprise delivering to the animal or human subject a heterogenous nucleic acid expressing the recombinant polypeptide; and allowing expression of the recombinant polypeptide to be expressed in the animal or human subject, thereby delivering to the animal or human subject an effective amount of the agent specifically interacting with a ligand of a TREM-like transcript 2 (TLT2) transmembrane receptor of a neutrophil or a macrophage

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following embodiments.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

Isolation of Mouse Neutrophils:

C57BL/6 mice 8-10 wk of age were used for isolation of bone marrow cells from the tibias and femurs. The bone marrow was passed through 25 and 20 gauge needles (Becton Dickinson, Franklin Lakes, N.J.) to generate single-cell suspensions in 1× HBSS (136 mM NaCl, 5.55 mM Glucose, 5.36 mM KCl, 4.16 mM NaHCO₃, 1.66 mM KH₂PO₄, 0.338 mM Na₂HPO₄, pH 7.2). Red blood cells were lysed by incubation in AKC (0.15 M NH₄Cl, 12 mM NaHCO₃, 0.1 mM EDTA, pH 7.2) for 5 min on ice, leukocytes were then separated by density sedimentation using a Percoll (Amersham, Piscataway, N.J.) gradient (60%/80%) in 1× HBSS centrifuged at room temperature at 1500 rpm for 25 min. The cells at the 60/80 interface, which was comprised of more than 95% neutrophils as assayed by flow cytometry, were collected, counted with a hemocytometer, and resuspended in media at appropriate concentrations. The media used in all experiments was RPMI-1640, supplemented with pen/strep, sodium pyruvate, mercaptoethanol, L-glutamine, and 5% FBS. Anti-CD11b-FITC (BD Pharmingen, San Diego, Calif.), anti-Gr-1-PE and anti-Gr-1-APC mAbs (Southern Biotech, Birmingham, Ala.) were used to determine the purity of isolated neutrophils.

### Example 2

### Respiratory Burst Assay

The αTLT2 mAbs 1H4 and 1C5 were generated as previously described (King et al., (2006) J. Immunol. 176: 6012, incorporated herein by reference in its entirety) and were prepared under LPS-free conditions. The antibody preparations used in these studies were subjected to the limulus amoebocyte lysate test, and were demonstrated to contain no detectable endotoxin (limit of detection is 0.03 EU/mL). Where indicated, antibodies were biotinylated using EZ-Link NHS-LC-biotin (Pierce, Rockford, Ill.). Following isolation, 1×10⁶ purified neutrophils in 100 μl of RPMI-1640 were incubated with αTLT2 mAb, isotype control mAb, LPS, or were left untreated for the specified times. Extracellular ROS scavengers (2000 units/ml catalase, Worthington, Lakewood, N.J.) and 50 units/ml superoxide dismutase (Sigma; St. Louis, Mo.) and 10% luminol (final concentration 5 μM) were added to the samples. After a 5 min equilibration period at 37° C., FMLP or GM-CSF (Calbiochem, Darmstadt, Germany) was added and the respiratory burst response was monitored using an Envision Multi-label Plate reader (Perkin Elmer, Waltham, Mass.) in the ultrasensitive luminescence mode.

### Example 3

### Neutrophil Degranulation Assay

A total of 1×10⁶ neutrophils per sample in 1 ml of RPMI-1640 were incubated with the appropriate αTLT2 mAb, isotype control mAb or medium alone as indicated for 10 min at 37° C. Following this incubation the FMLP agonist, WKYMVm (Calbiochem) C5a, GM-CSF or the TLR agonists LPS (Sigma), monophosphoryl lipid A (MPLA) (Dr. John Kearney, Microbiology, UAB), Poly(I:C) (Invitrogen), flagellin (Dr. Charles Elson, Medicine, UAB), imiquimod (Invitrogen) and CpG-ODN (Invitrogen) were added at the indicated concentrations for the indicated times. After stimulation, all reactions were terminated by addition of ice cold 1×PBS. To examine the effect of secondary cross-linking of TLT2, 1 μg of biotinylated αTLT2 mAb was pre-incubated with varying concentrations (2-40 μg) of streptavidin (Pierce), which was then added to the neutrophil preparations for 10 min prior to the addition of triggering stimuli. After the indicated period of time, the neutrophils were then stained for the surface markers CD11b and Gr-1. Degranulation was measured by a specific increase in the cell surface expression of CD11b.

### Example 4

### Phagocytosis Assay

A total of 5×10⁶ 2.0 micron biotinylated polystyrene beads (Polysciences, Inc, Warrington, Pa.) were incubated with a 1:100 dilution of streptavidin-FITC (Biosource, Carlsbad, Calif.) in 100 μl PBS for 15 min at room temperature in the dark. The beads were then washed 3 times with 1×PBS. Subsequently, 50% of the FITC-labeled beads were incubated at room temperature with mouse serum containing anti-FITC antibodies for 30 min in the dark. The FITC-specific antiserum was generated by immunizing C57BL/6 mice with FITC conjugated protein antigen, after which serum was harvested on day 10. After opsonization with the antiserum, the beads were washed 3 times with 1×PBS and warmed to 37° C. prior to addition to neutrophils. Neutrophils (1×10⁵ cells/sample) in 500 μl RPMI-1640 were pre-incubated with the indicated stimuli for 10 min. 1×10⁶ fluorescent beads were then introduced to the cell suspensions followed by a 15 min incubation. Trypan blue was added to quench extracellular FITC fluorescence and phagocytosis of the beads was assayed by flow cytometry.

### Example 5

### Chemotaxis Assay

Neutrophil chemotaxis was assayed using 3.0 micron, 6.5 mm transwell inserts (Costar, Corning, N.Y.) placed in 24-well cell culture plates. 1×10⁶ neutrophils were placed in 100 μl of RPMI-1640 in the upper chamber and the chemotactic factors FMLP, MIP-2, KC, IL-8, C5a, or GM-CSF were added at the indicated concentrations to 600 μl in the bottom chamber in 37° C. RPMI-1640. The murine recombinant proteins KC, MIP-2, GM-CSF and human recombinant IL-8 were obtained from PeproTech (Rocky Hill, N.J.), and murine C5a was obtained from eBioscience (San Diego, Calif.). αTLT2 or isotype control mAb was added to the upper chamber with the neutrophils at specified concentrations, and the samples were incubated for 60 min at 37° C. in a humidity-controlled incubator under 5% CO₂. Migrating neutrophils were removed from the bottom chamber and counted.

The normalized chemotactic index was calculated by dividing the number of cells migrating in samples containing αTLT2 or isotype control mAbs by the number migrating in response to the chemotactic agent alone.

Purified neutrophils (1×10⁶) were placed in the upper chamber of a 3 μm transwell device in the presence or absence of the indicated concentration of the αTLT2 mAb 1H4 (FIG. 7A) or 100 μg of either intact or F(ab′)₂ fragments of 1H4 (FIG. 7B) or 1 μg of the αTLT2 mAb 1C5 coupled to biotin with or without streptavidin (20 μg) (C). For panels FIG. 7B and FIG. 7C, isotype control indicates samples in which 0.1 or 1 μg/ml of rat mAb was added to the upper chamber, respectively. For panels (FIGS. 7A-7C) 1 μM FMLP was added to the lower chamber for all samples. After 1 h, the cells were harvested from the lower chamber and counted. The number of cells present in the lower chamber for each experimental condition was divided by the number of migrating cells in control samples (FMLP alone) to determine the chemotactic index. The mean±SD shown for triplicate samples and the experiments depicted are representative of at least 3 independent experiments. (FIG. 7D) TLT2 ligation enhances neutrophil migration in response to a wide range of FMLP concentrations. Neutrophils (1×10⁶) in medium alone, or containing 100 ng/ml of 1H4 mAb or 2 μg/ml LPS were placed in the upper chambers of transwell devices. The indicated concentrations of FMLP or medium alone were placed in the lower chambers. The percentage of cells migrating into the lower chamber are the average of triplicate samples with the mean±SD shown, and are representative of at least three independent experiments. (FIG. 7E) TLT2 potentiation of FMLP-mediated chemotaxis is proportional across a wide dose response. The chemotactic indices for αTLT2 mAb pretreated versus control neutrophils in response to FMLP are depicted. The values are derived by dividing the % migration data for αTLT2 pretreated samples by the % migration for neutrophils incubated in medium alone as shown in (FIG. 7D). Asterisks denote significance of values for αTLT2 mAb-treated samples compared to the respective control sample.

### Example 6

### Ear Model of Inflammation

To induce inflammation in the ear, 20 μl of 2% croton oil (Sigma, St. Louis, Mo.) in acetone was applied to both sides of the pinna of one ear, whereas acetone alone was applied to the control ear. Either 100 μg of αTLT2 mAb or 1×PBS alone was injected intravenously (i.v.) into mice 30 min prior to the application of croton oil. Four hours following the application of croton oil, 4 mm biopsies were obtained from the proximal portion of the ear, mechanically homogenized with a Tissue Tearor homogenizer (Biospec Products, Bartlesville, Okla.), and assayed for MPO activity.

MPO levels were determined by the addition of the chromogenic substrate 3,3′,5,5′-tetramethylbenzidine (TMB) (Sigma) to 100 μl of the ear lysate. The absorbance at 450 nm was then measured using a Vmax plate reader (Molecular Devices, Sunnyvale, Calif.). These samples were assayed with a standard curve of known concentrations of the enzyme HRP. Comparing the MPO activity present in specific numbers of purified neutrophils to this standard curve allowed for the estimation of the number of neutrophils present in the biopsy samples.

To examine the effect of administration of αTLT2 mAb on the recruitment of cells and architecture of the ear, mice were sacrificed and the ears were removed, fixed overnight in 10% NBF, paraffin embedded, and 8 micron thick sections prepared. The sections were treated to remove the paraffin and rehydrated, after which they were stained with H&E (Harris Hematoxylin and Eosin, Sigma). The stained sections were subsequently dehydrated, cleared, and mounted with Permount (Fisher #SP15). The slides were examined with a Zeiss AX10 microscope using a 20× objective and pictures were taken using a Zeiss AxioCam MRC.

### Example 7

### Lung Model of Inflammation and Adoptive Transfer of Neutrophils

To induce inflammation in the lung 5 μg of LPS was introduced intratracheally. Bone marrow neutrophils were isolated from CD45.1 donor mice. Once isolated, the neutrophils were divided equally into 3 groups of 3×10⁷ neutrophils and subsequently incubated for 10 min in 500 μl of 1×PBS containing either 5 μM CSFE, 5 μM Cell Tracker CMPTX Red or without fluorescent additives. CSFE and CMTPX cell permeable tracking dyes were obtained from Invitrogen (Carlsbad, Calif.). Each group of neutrophils was then subjected to the indicated treatment for 15 min at 37° C., followed by 3 washes in pre-warmed 1×PBS and resuspended in 100 μl. After loading, the cells were mixed in a 1:1:1 ratio and approximately 3×10⁷ total neutrophils in 100 μl were injected intravenously into CD45.2 mice 2 h after the initiation of lung inflammation. After an additional 4 h, mice were sacrificed and cells were isolated from lungs by bronchoalveolar lavage. The isolated cell suspensions were stained with anti-CD45.1-APC (eBioscience). Flow cytometry was performed to identify CD45.1 expressing cells and neutrophils subjected to the indicated treatment conditions were then resolved based on CSFE or CMTPX fluorescence, or the absence of fluorescent labeling.

### Example 8

### In Vivo Models of Inflammation and Cytokine Analysis

C57Bl/6 mice 8-10 wk of age were used in all experiments. Mice were given i.v. injections of PBS, or anti-TLT2 F(ab′)₂ mAb (1H4 or 1C5, 50-100 μg) 30 min prior to the addition of 20 μl of 2% croton oil in acetone, or 20 μl of acetone alone to the control ear. Immediately after sacrifice, 4 mm ear biopsies were taken from the center of each ear with a biopsy punch and homogenized with a Tissue Tearor homogenizer (Biospec Products, Bartlesville, Okla.). The ear homogenates were prepared in PBS supplemented with Complete Mini Protease inhibitor tablets (Roche), clarified by centrifugation, and stored at −80° C. The resulting homogenates were analyzed using the Milliplex 32 Plex Chemokine/Cytokine Panel (Premix—MCYTMAG-70 KPX32, Millipore, Billirica, Mass.) using a Luminex-based Bio-Plex multiplex suspension protein array (Bio-Rad Laboratories, Hercules, Calif.), according to the manufacturer's instructions. Concentrations of each cytokine and chemokine were determined using Bio-Plex Manager version 4.1.1 software. Myeloperoxidase (MPO) activity in homogenates was analyzed following the addition of the chromogenic substrate 3,3,5,5′-tetramethylbenzidine (TMB) (Sigma, St. Louis, Mo.). To determine the specific in vivo effects attributed to administration of anti-TLT2 mAb, F(ab′)₂ or isotype control (IC) Ab (clone KLH/G2a-1-1, SouthernBiotech, Homewood, Ala.) fragments were injected either i.v., i.p., or intratrachealy (i.t.). Peritoneal or bronchial lavage fluid was collected and subjected either to Bio-plex or flow cytometric analysis at the indicated time points to quantitate soluble factors or leukocyte populations, respectively.

### Example 9

### Ex Vivo Treatment of Macrophages and Cytokine Analysis

Resident peritoneal macrophages were purified by adhesion to culture dishes and a total of 1×10⁵ macrophages per sample were seeded in 96-well, flat-bottom microtiter plates in 100 μl of medium and were incubated for 2 h at 37° C., 5% C0₂. Following this, 100 of medium containing either 10 μg/ml of anti-TLT2 F(ab′)₂ mAb, 10 μg/ml IC Ab, or medium alone was added and the plates were incubated at 37° C., 5% C0₂ for up to 4 h. Aliquots of the supernatants were collected at 30 min, 2 h, and 4 h for Bio-plex analysis.

### Example 10

### Flow Cytometry

Neutrophils were identified using anti-CD11b-FITC (BD Pharmingen; San Diego, Calif.) and anti-Gr-1-PE (Southern Biotech; Birmingham, Ala.). Peritoneal macrophages were identified based on forward and side scatter, anti-CD11b-allophycocyanin and anti-F4/80-488. Macrophage activation was assayed using anti-CD80-PE-cy5.5, anti-CD86-FITC, and anti-CD69-PE all purchased from Becton Dickinson. 7-Aminoactinomycin D (7-AAD) was used to discriminate non-viable cells. Cells were washed three times at 4° C. with FACS buffer after a 15 min, 4° C. incubation with Ab in 96-well microtiter plates prior to flow cytometric analysis. Samples were analyzed immediately either on a FACSCalibur or LSR II flow cytometer (BD Biosciences), and data were analyzed using FlowJo (Tree Star; Ashland, Oreg.).

Cells were washed three times with FACS buffer (1×PBS, 0.01% NaN₃⁺, 0.5% FBS) and then incubated with the appropriate fluorescent Ab mixture in 96-well microtiter plates for 15 min on ice. After this incubation, the cells were again washed three times with FACS buffer. When necessary, samples were incubated with secondary antibodies for an additional 15 min on ice. Samples were analyzed immediately following labeling on either a FACScan. FACSCalibur or LSR II flow cytometer (BD Biosciences), and the resultant data were analyzed using FlowJo (Tree Star, Ashland Oreg.

### Example 11

### Immunohistochemistry

Mice were administered either PBS or anti-TLT2 mAb (1H4 F(ab′)₂) by i.v. injection. After 30 min, croton oil (2% in acetone) was applied to one ear versus acetone alone to the control ear. After 12 h, 4 mm biopsies were obtained from the ears of PBS control and anti-TLT2 mAb treated mice. The tissue specimens were placed in 10% neutral buffered formalin overnight. Following fixation, the specimens were paraffin embedded, 8 mm sections were cut and then were stained with H&E. Images were obtained using a Zeiss AX10 microscope with a Ziess EcPlan 10× lens and captured using an AxioCam MRc camera. Image capture and processing was performed using Axiovision 4.5 software. White Balance, contrast and gamma enhancements were performed using Photoshop 7 software.

### Example 12

### Statistical Analysis

Significance values for neutrophil recruitment and Bio-plex experiments were calculated in Prism (GraphPad Software. La Jolla. CA) using the Mann-Whitney test. The p-value of significant differences is reported, with a p-value less than or equal to 0.05 considered statistically significant.

Statistical analyses were computed via the student two-tailed t-test. The CD45.1 adoptive transfer experiment was analyzed with the one-way anova. The p-value of significant differences is reported, with a p-value <0.05 considered statistically significant.

### Example 13

### Chemotaxis Assay

Purified neutrophils (1×10⁶) were placed in the upper chamber of a 3 μm transwell device in the presence or absence of the indicated concentration of the αTLT2 mAb 1H4 (as shown in FIG. 7A) or 100 μg of either intact or F(ab′)₂ fragments of 1H4 (as shown in FIG. 7B) or 1 μg of the αTLT2 mAb 1C5 coupled to biotin with or without streptavidin (20 μg) (as shown in FIG. 7C). As shown in FIGS. 7B and 7C, isotype control indicates samples in which 0.1 or 1 μg/ml of rat mAb was added to the upper chamber, respectively. For FIGS. 7B and 7C, 1 μM FMLP was added to the lower chamber for all samples. After 1 h, the cells were harvested from the lower chamber and counted.

The number of cells present in the lower chamber for each experimental condition was divided by the number of migrating cells in control samples (FMLP alone) to determine the chemotactic index. The mean±SD is shown for triplicate samples and the experiments depicted were representative of at least 3 independent experiments.

As shown in FIG. 7D, TLT2 ligation enhanced neutrophil migration in response to a wide range of FMLP concentrations. Neutrophils (1×10⁶) in medium alone, or containing 100 ng/ml of 1H4 mAb or 2 μg/ml LPG were placed in the upper chambers of transwell devices. The indicated concentrations of FMLP or medium alone were placed in the lower chambers. The percentage of cells migrating into the lower chamber are the average of triplicate samples with the mean±SD shown, and are representative of at least three independent experiments.

As shown in FIG. 7E, TLT2 potentiation of FMLP-mediated chemotaxis is proportional across a wide dose response. The chemotactic indices for αTLT2 mAb pretreated versus control neutrophils in response to FMLP are depicted. The values are derived by dividing the % migration data for αTLT2 pretreated samples by the % migration for neutrophils incubated in medium alone as shown in FIG. 7E. Asterisks denote significance of values for αTLT2 mAb-treated samples compared to the respective control sample.

### Example 14

### Neutrophil Degranulation Assay

Purified neutrophils (1×10⁶) were incubated either with the αTLT2 mAb 1H4 (at the concentrations indicated) or biotinylated 1C5 (1 μg/ml). Where indicated, streptavidin (2-40 μg) was added to mediate secondary cross-linking of TLT2. Neutrophils were stimulated with the FMLP agonist WKYMVm (W) at the indicated concentrations (FIGS. 8A-8F), the activated complement component C5a (2 ng/ml) (G), or GM-CSF (1 ng/ml) (FIGS. 8G-8I). At the indicated time points, the cells were harvested and stained to detect surface expression of CD11b and analyzed by flow cytometry.

### Example 15

### Enhanced Migration of Neutrophils

Purified neutrophils were subjected to migration assays using 3 μm transwell filters. Neutrophils (1×10⁶) were placed in the upper chamber in medium alone (control) or in the presence of the indicated concentration of αTLT2 mAb. Medium alone (control) or containing chemoattractants (FMLP, 1 μM; MIP-2, 5 ng/ml; KC, 5 ng/ml; IL-8, 50-100 ng/ml or GM-CSF, 0.5-10 ng/ml) was placed in the lower chamber, as indicated. After 1 h incubation at 37° C., the neutrophils present in the lower chamber were harvested and counted. The percentage of cells present in the lower chamber (as shown in FIG. 10A) and the chemotactic index is shown in FIG. 10B. The chemotactic index was calculated by dividing the % migration for neutrophils incubated with each chemoattractant in the presence or absence of αTLT2 mAb by the % migration for neutrophils incubated with medium alone. Data represent the average of triplicate samples with the mean±SD shown.

### Example 16

### Enhanced Accumulation of Neutrophils at Sites of Inflammation In Vivo

Either 100 μg of the αTLT2 mAb 1H4 or an equivalent volume of PBS was administered to groups of mice via i.v. injection. After 30 min, a solution of 2% croton oil in acetone was applied to the pinna of one ear, whereas only acetone was applied to the other (control ear). After 4 h, the mice were sacrificed and a 4 mm biopsy was removed from the center of the ears using a biopsy punch. These samples were homogenized and assayed for MPO activity using the colorimetric substrate TMB. The measured absorbance values are depicted for αTLT2 mAb treated and control mice for both ears (as shown in FIG. 11A). To determine the number of neutrophils recruited, lysates from known numbers of purified neutrophils were compared to serial dilutions of HRP to develop a standard curve. Using this standard curve for the MPO dependent conversion of TMB, the numbers of neutrophils present in the croton oil treated ears for αTLT2 mAb treated and control mice were calculated (as shown in FIG. 11B). The data presented represent a minimum of 5 mice and the mean±SD, and significant p values are shown for both FIG. 11A and FIG. 11B. FIG. 11C shows the H & E staining of ear sections taken at 3 h from mice injected with saline (control) or αTLT2 mAb. Representative sections are shown for both control ears that were treated with acetone alone as well as inflamed ears treated with croton oil in acetone. The sections are representative of at least three independent experiments.

### Example 17

### TLT2 Ligation Enhances Neutrophil Recruitment into Sites of Inflammation In Vivo

FIG. 12A shows purified neutrophils from CD45.1 mice labeled with either CFSE, CMPTX, or left unlabeled. These cells were then incubated in the presence of the αTLT2 mAb as indicated in FIG. 12A, or an isotype control antibody, or medium alone. After treatment, the purified, labeled neutrophils were mixed at equivalent ratios and 3×10⁷ total cells were adoptively transferred into CD45.2 recipient mice, which had received an intratracheal challenge with 5 μg of LPS to induce inflammation in the lung 30 min prior to adoptive transfer. After 4 h, the recipient mice were sacrificed and the neutrophils present in the lung were isolated by lavage.

Donor derived neutrophils were identified based on the expression of CD45.1, and these cells were analyzed for the presence of the fluorescent dyes, which discriminated the pre-transfer treatment conditions.

FIG. 12B shows ligation of TLT2 increases the relative frequency of neutrophils recruited into the lungs of recipient mice. A total of 8 mice are depicted and for each mouse the percentage PMNs recruited to the lung for each of 3 pre-treatment conditions is shown. TLT2 ligation causes an absolute increase in the number of neutrophils that migrate into inflamed lungs. The absolute number of neutrophils for each treatment condition was calculated based on flow cytometric analysis. The mean±SD for a minimum of 5 mice is shown for each treatment condition.

### Example 18

### Anti-TLT2 mAb-Mediated Protection Against Challenge with S. pneumonia

Mice were given an intratracheal challenge with 1×10⁶ S. pneumonia. At the time of challenge, one group of mice (n=7) were given an intravenous injection of 100 μg of isotype control mAb (IC), and a second group (n=7) were given an intravenous injection of 100 μg of anti-TLT2 mAb (1H4). Mice were observed for a period of 250 hours. Whereas mice that received anti-TLT2 mAb did not become moribund or die, 50% of the mice that received IC mAb were moribund or deceased at 250 hours. Thus, administration of anti-TLT2 mAb provides protection against lethal challenge with S. pneumonia.

### Example 19

### Administration of a TLT2 Extracellular Domain-Fc Region Fusion Protein Reduces an Inflammatory Response

Support for TLT2 playing a critical role in regulating the innate immune response and inflammation has been generated by experiments in which a recombinant TLT2 fusion protein consisting of the extracellular domain of TLT2 fused in frame with the Fc region of human IgG₁ and having the amino acid sequence SEQ ID No.: 1 as shown in FIG. 15 was injected intravenously into mice in conjunction with induction of an inflammatory response in the lung following intratracheal administration of LPS.

As seen in FIG. 14, significant neutrophil recruitment into inflamed lungs was observed in mice that received a control intravenous injection. In contrast, neutrophil recruitment was significantly reduced by 60-70% in mice that received the recombinant TLT2:Fc fusion protein. These data provide significant evidence to support the conclusion that binding of an endogenous ligand to TLT2 plays a critical role in driving neutrophil recruitment to sites of inflammation in vivo. It is equally likely that a similar mechanism is involved in the response to microbial pathogens.

What is claimed:

1. A method for enhancing an innate immune response of an animal or human subject, the method comprising the steps of delivering to a cell or population of cells an effective amount of an agent that specifically interacts with a TREM-like transcript 2 (TLT2) transmembrane receptor of the cell or population of cells and potentiates neutrophil and/or macrophage activation and/or migration.

2. The method of claim 1, wherein the cell or population of cells is a neutrophil or population of neutrophils, wherein said cell or population of cells are isolated from an animal or human subject, a cultured neutrophil or population of neutrophils; or a neutrophil or population of neutrophils in an animal or human subject.

3. The method of claim 1, wherein the cell or population of cells is a macrophage or population of macrophages, wherein said cell or population of cells are isolated from an animal or human subject, a cultured macrophage or population of macrophages; or a macrophage or population of macrophages in an animal or human subject.

4. The method of claim 1, wherein the agent is an antibody, or a fragment thereof, capable of specifically binding to an extracellular domain of TLT2 of a neutrophil or a macrophage.

5. The method of claim 4, wherein the antibody is a monoclonal antibody or a fragment thereof.

6. The method of claim 1, wherein the agent delivered to the cell or population of cells initiates ligation of the TLT2 thereon.

7. The method of claim 1, wherein the agent delivered to the cell or population of cells induces the release of at least one cytokine by the cell or population of cells, wherein said at least one cytokine is characterized as capable of interacting with at least one G-protein coupled receptor of a neutrophil, thereby inducing neutrophil activation and/or migration.

8. The method of claim 1, wherein the agent delivered to the cell or population of cells is mixed with a pharmaceutically acceptable carrier.

9. A method of potentiating a neutrophil response to a G-protein coupled receptor signaling comprising delivering to a neutrophil an effective amount of an agent specifically interacting with TREM-like transcript 2 (TLT2) transmembrane receptor of the neutrophil.

10. The method of claim 9, wherein the neutrophil is an isolated neutrophil, a cultured neutrophil, or is a neutrophil of an animal or human subject.

11. A method of modulating an inflammatory response of an animal or human comprising delivering to the animal or human subject a composition comprising an effective amount of an agent specifically interacting with a ligand of a TREM-like transcript 2 (TLT2) transmembrane receptor.

12. The method of claim 11, wherein the agent is a recombinant polypeptide comprising a domain specifically binding to a TLT2-binding ligand.

13. The method of claim 12, wherein the domain specifically binding to a TLT2 agonist is an extracellular region of TLT2, or a fragment thereof.

14. The method of claim 11, wherein the agent delivered to the animal or human subject is mixed with a pharmaceutically acceptable carrier.

15. The method of claim 11, wherein the method further comprises delivering to the animal or human subject a heterogenous nucleic acid expressing the recombinant polypeptide; and allowing expression of the recombinant polypeptide to be expressed in the animal or human subject, thereby delivering to the animal or human subject an effective amount of the agent specifically interacting with a ligand of a TREM-like transcript 2 (TLT2) transmembrane receptor of a neutrophil or a macrophage
